(19) United States
(12) Patent Application Publication
(10) Pub. No.: US 2026/0092297 A1
(43) Pub. Date: Apr. 2, 2026

(54) NOVEL PROMOTER AND 5'-UNTRANSLATED REGION MUTATIONS ENHANCING PROTEIN PRODUCTION IN GRAM-POSITIVE CELLS

(71) Applicant: DANISCO US INC., Palo Alto, CA (US)

(72) Inventors: Frits Goedegebuur, Vlaardingen (NL); Cristina Bongiorni, Fremont, CA (US); Ryan L. Frisch, Newark, DE (US); Harm Mulder, Voorhout (NL)

(21) Appl. No.: 19/106,952

(22) PCT Filed: Sep. 1, 2023
(86) PCT No.: PCT/US2023/073280

§ 371 Date: Feb. 26, 2025

Related U.S. Application Data
(60) Provisional application No. 63/374,450, filed on Sep. 2, 2022.

(51) Int. Cl. C12P 21/00; C12N 15/75

(52) U.S. Cl. CPC C12P 21/00; C12N 15/75; C12N 2800/101

(57) ABSTRACT

The present disclosure is generally related to the fields of microbial host cells, molecular biology, protein engineering, fermentation, protein production, and the like. Certain aspects of the disclosure are related to novel promoter and 5′-untranslated region a nucleic acid (DNA) sequences.

### CROSS-REFERENCE TO RELATED APPLICATIONS

This application claims priority to U.S. Provisional Patent Application No. 63/374,450 filed Sep. 2, 2022, the disclosure of which is herein incorporated by reference in their entirety.

### FIELD

The present disclosure is generally related to the fields of microbial host cells, molecular biology, protein engineering, fermentation, protein production, and the like. Certain aspects of the disclosure are related to novel promoter and 5′-untranslated region nucleic acid (DNA) sequences.

### REFERENCE TO A SEQUENCE LISTING

The contents of the electronic submission of the text file Sequence Listing, named “NB41861-WO-PCT_SequenceListing.xml” was created on Aug. 30, 2023 and is 53 KB in size, which is hereby incorporated by reference in its entirety.

### BACKGROUND

Genetic engineering has facilitated various improvements in host microorganisms used as industrial bioreactors or cell factories. For example, Gram-positive bacterial host cells can produce and secrete a large number of useful proteins and metabolites. The most common Bacillus sp. used in industry are B. licheniformis, B. amyloliquefaciens, and B. subtilis. Because of their generally recognized as safe (GRAS) status, strains of Bacillus sp. are natural candidates for the production of proteins utilized in the food and pharmaceutical industries. For example, important production enzymes include α-amylases, neutral proteases, alkaline (or serine) proteases, and the like. However, in spite of advances in the knowledge of production of proteins in Bacillus host cells, there remain needs for methods and compositions thereof which improve the expression/production of these proteins by microorganisms.

Recombinant production of a protein of interest (POI) encoded by a gene (or ORF) of interest is typically accomplished by constructing expression vectors suitable for use in a desired host cell, wherein the nucleic acid encoding the desired POI is placed under the expression control of a promoter. Thus, the expression vector is introduced into a host cell by various techniques (e.g., via transformation), and production of the desired protein product is achieved by culturing the transformed host cell under conditions suitable for the expression and production of the protein product. For example, Bacillus sp. promoters (and associated elements thereof) for the recombinant expression of functional polypeptides have been described (e.g., Kim et al., 2008 and U.S. Pat. No. 4,559,300). While numerous promoters are known, there remains a need in the art for novel promoter (nucleic acid) sequences which can improve the expression of heterologous nucleic acids encoding proteins of interest. For example, in the industrial biotechnology arts, even relatively small increases in the expression/productions levels of an industrially relevant protein (e.g., an enzyme, an antibody, a receptor, and the like) translate into significant cost, energy and time savings of the recombinant protein produced.

### SUMMARY

As generally described herein, the instant disclosure provides, inter alia, compositions and methods for the production of proteins of interest in Gram-positive bacterial (host) cells. Certain embodiments are related to novel promoter and 5′-UTR nucleic acid (DNA) sequences, recombinant polynucleotides (e.g., vectors, plasmids, expression cassettes, etc.) comprising novel promoter/5′-UTR sequences, recombinant polynucleotides comprising novel promoter/5′-UTR sequences operably linked to DNA sequences encoding protein signal (secretion) sequences, and/or operably linked to DNA sequences encoding pro-region sequences, operably linked to DNA sequences encoding proteins of interest and the like.

Certain embodiments of the disclosure therefore provide variant nucleic acid sequences comprising at least one mutation set forth in any one of SEQ ID NO: 8 through SEQ ID NO: 46, wherein the nucleotide positions of the variant nucleic acid sequences are numbered according to SEQ ID NO: 1. In certain other embodiments, a variant nucleic acid comprises the nucleotide sequence of any one of SEQ ID NO: 8 through SEQ ID NO: 46, wherein the nucleotide positions of the variant sequences are numbered according to SEQ ID NO: 1. In certain other embodiments, variant nucleic acid sequences comprise at least about 97.5% identity to SEQ ID NO: 1. In related aspects, variant nucleic acid sequences of the disclosure may be referred to as variant promoter/5′-untranslated region (5-UTR) sequences.

Certain other one or more embodiments are related to polynucleotides (DNA) comprising variant nucleic acid sequences of the disclosure. Certain embodiments therefore provide polynucleotides comprising variant nucleic acid sequences of the disclosure operably linked to downstream (3′) nucleic acid sequences encoding proteins of interest.

In certain other embodiments or aspects, polynucleotides comprising a variant nucleic acid sequence of the disclosure are operably linked to a downstream (3′) nucleic acid sequence encoding Pro-region sequence operably linked to a downstream nucleic acid sequence encoding a mature protein of interest (POI). In certain other embodiments or aspects, polynucleotides comprising a variant nucleic acid sequence of the disclosure are operably linked to a downstream (3′) nucleic acid sequence encoding a protein signal (secretion) sequence (SS) operably linked to a downstream nucleic acid sequence encoding a mature protein of interest (POI). In certain other embodiments or aspects, polynucleotides comprising a variant nucleic acid sequence of the disclosure are operably linked to a downstream (3′) nucleic acid sequence encoding a protein signal (secretion) sequence (SS) operably linked to a downstream nucleic acid sequence encoding Pro-region (PRO) sequence operably linked to a downstream nucleic acid sequence encoding a mature protein of interest (POI).

In certain related embodiments, a POI is selected from the group consisting of enzymes, antibodies, receptor proteins, lectins and regulatory proteins.

Other embodiments provide expression cassettes comprising polynucleotides of the instant disclosure. In related embodiments, Gram-positive bacterial (host) cells comprise one or more introduced cassettes of the disclosure. In certain embodiments, a Gram-positive host cell is a Bacillus sp. cell. In related embodiments, a Bacillus sp. (host) cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis.

Certain other embodiments of the disclosure provide methods for producing a protein of interest (POI) in a Gram-positive bacterial cell comprising (a) introducing into a Gram-positive cell an expression cassette comprising a variant nucleic acid sequence of any one of SEQ ID NO: 8 through SEQ ID NO: 46 operably linked to a downstream (3′) nucleic acid sequence encoding a protein of interest (POI), and (b) cultivating the modified cell under suitable conditions for the production of the POI. In certain preferred embodiments of the methods, the modified cell produces an increased amount of the POI relative to (vis-à-vis) a control Gram-positive cell comprising an introduced expression cassette comprising the reference nucleic acid sequence of SEQ ID NO: 1 operably linked to a downstream (3′) nucleic acid sequence encoding the same POI, wherein the modified and control cells are cultivated under the same conditions.

In certain other embodiments of the methods, the modified cells produce an increased amount of the POI relative to the control cell after at least about seventy-two (72) hours of cultivation. In yet other embodiments of the methods, a protein of interest (POI) is selected from the group consisting of enzymes, antibodies, receptor proteins, lectins and regulatory proteins. In certain embodiments of the methods, enzymes are selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof. In certain other embodiments of the methods, a Gram-positive bacterial cell is a Bacillus sp. cell.

### BRIEF DESCRIPTION OF DRAWINGS

FIG. 1 presents the nucleotide sequence of a variant rrnI-P2 promoter/5′-UTR region DNA sequence (SEQ ID NO: 1). More particularly, as presented in FIG. 1, the variant (reference) rrnI-P2 promoter/5′-UTR region sequence comprises nucleotide positions 1 to 149 of SEQ ID NO:1, wherein the “UP”, “−35”, “−10” and “Shine-Dalgarno” sequence elements are indicated with bold text.

FIG. 2 presents DNA sequence alignments of the reference variant rrnI-P2 promoter/5′-UTR (SEQ ID NO: 1) and certain SEL variant promoter/5′-UTR region sequences of the disclosure. More particularly, as shown in FIG. 2A and FIG. 2B, nucleotide positions of the SEL variant sequences described in the Examples are aligned with the reference rrnI-P2 promoter/5′-UTR region (SEQ ID NO: 1; nucleotide positions 1-149). As shown in the FIG. 2A, nucleotide positions 1-83 of the reference rrnI-P2 promoter region include the UP element, −35 and −10 elements indicated with grey shading, and as shown in FIG. 2B, nucleotide positions 83-149 of the reference rrnI-P2 promoter/5′-UTR region include the Shine-Dalgarno element indicated with grey shading. As presented in FIG. 2A, modified nucleotide positions of the rrnI-P2 promoter region are indicated with black-shadowed nucleotide residues (e.g., FIG. 2A, UTR-00664; TGA).

FIG. 3 presents DNA sequence alignments of the reference variant rrnI-P2 promoter/5′-UTR region (SEQ ID NO: 1) and certain SEL variant promoter/5′-UTR region sequences of the disclosure. More particularly, as shown in the FIG. 3A and FIG. 3B, nucleotide positions of the SEL variant sequences described in the Examples are aligned with the reference rrnI-P2 promoter/5′-UTR region (SEQ ID NO: 1; nucleotide positions 1-149). As shown in the FIG. 3A, nucleotide positions 1-83 of the reference rrnI-P2 promoter/5′-UTR region include the UP element, −35 and −10 elements indicated with grey shading, and as shown in the FIG. 3B, nucleotide positions 83-149 of the reference rrnI-P2 promoter/5′-UTR region include the Shine-Dalgarno element indicated with grey shading. As presented in FIG. 3A/3B, modified nucleotide positions of the rrnI-P2 promoter region are indicated with black-shadowed nucleotide residues (e.g., FIG. 3A, UTR-00798; A).

### BRIEF DESCRIPTION OF THE BIOLOGICAL SEQUENCES

SEQ ID NO: 1 is a nucleic acid (DNA) sequence of a variant (reference) rrnI-P2 promoter/5′-UTR region.

SEQ ID NO: 2 is the amino acid sequence of a wild-type Bacillus gibsonii subtilisin named “BG46”.

SEQ ID NO: 3 is the amino acid sequence of a variant B. gibsonii BG46 subtilisin named BG46_variant”.

SEQ ID NO: 4 is a DNA sequence encoding a wild-type B. subtilis AprE protein signal sequence.

SEQ ID NO: 5 is a DNA sequence encoding a wild-type B. lentus pro-region sequence.

SEQ ID NO: 6 is a DNA sequence of a wild-type B. amyloliquefaciens BPN′ terminator.

SEQ ID NO: 7 is the DNA sequence of the kanamycin (kan) gene expression cassette.

SEQ ID NO: 8 is the DNA sequence of variant UTR-00664.

SEQ ID NO: 9 is the DNA sequence of variant UTR-00692.

SEQ ID NO: 10 is the DNA sequence of variant UTR-00330.

SEQ ID NO: 11 is the DNA sequence of variant UTR-00411.

SEQ ID NO: 12 is the DNA sequence of variant UTR-00325.

SEQ ID NO: 13 is the DNA sequence of variant UTR-00730.

SEQ ID NO: 14 is the DNA sequence of variant UTR-00348.

SEQ ID NO: 15 is the DNA sequence of variant UTR-00738.

SEQ ID NO: 16 is the DNA sequence of variant UTR-00788.

SEQ ID NO: 17 is the DNA sequence of variant UTR-00792.

SEQ ID NO: 18 is the DNA sequence of variant UTR-00800.

SEQ ID NO: 19 is the DNA sequence of variant UTR-01018.

SEQ ID NO: 20 is the DNA sequence of variant UTR-01112.

SEQ ID NO: 21 is the DNA sequence of variant UTR-00037.

SEQ ID NO: 22 is the DNA sequence of variant UTR-00039.

SEQ ID NO: 23 is the DNA sequence of variant UTR-00661.

SEQ ID NO: 24 is the DNA sequence of variant UTR-00891.

SEQ ID NO: 25 is the DNA sequence of variant UTR-00084.

SEQ ID NO: 26 is the DNA sequence of variant UTR-00362.

SEQ ID NO: 27 is the DNA sequence of variant UTR-00424.

SEQ ID NO: 28 is the DNA sequence of variant UTR-00643.

SEQ ID NO: 29 is the DNA sequence of variant UTR-00645.

SEQ ID NO: 30 is the DNA sequence of variant UTR-00741.

SEQ ID NO: 31 is the DNA sequence of variant UTR-00798.

SEQ ID NO: 32 is the DNA sequence of variant UTR-00960.

SEQ ID NO: 33 is the DNA sequence of variant UTR-01223.

SEQ ID NO: 34 is the DNA sequence of variant UTR-00656.

SEQ ID NO: 35 is the DNA sequence of variant UTR-00657.

SEQ ID NO: 36 is the DNA sequence of variant UTR-00030.

SEQ ID NO: 37 is the DNA sequence of variant UTR-01092.

SEQ ID NO: 38 is the DNA sequence of variant UTR-00721.

SEQ ID NO: 39 is the DNA sequence of variant UTR-00651.

SEQ ID NO: 40 is the DNA sequence of variant UTR-00301.

SEQ ID NO: 41 is the DNA sequence of variant UTR-00187.

SEQ ID NO: 42 is the DNA sequence of variant UTR-00035.

SEQ ID NO: 43 is the DNA sequence of variant UTR-00005.

SEQ ID NO: 44 is the DNA sequence of variant UTR-00863.

SEQ ID NO: 45 is the DNA sequence of variant UTR-00711.

SEQ ID NO: 46 is the DNA sequence of variant UTR-00752.

SEQ ID NO: 47 is the DNA sequence of the 5′ aprE gene FR.

SEQ ID NO: 48 is the DNA sequence of the 3′ aprE gene FR.

### DETAILED DESCRIPTION

As briefly set forth above and described hereinafter in detail, the present disclosure provides, inter alia, novel promoter and 5′-UTR nucleic acid (DNA) sequences, recombinant polynucleotides (e.g., vectors, plasmids, expression cassettes, etc.) comprising novel promoter/5′-UTR sequences, recombinant polynucleotides comprising novel promoter/5′-UTR sequences operably linked to DNA sequences encoding protein signal (secretion) sequences, and/or operably linked to DNA sequences encoding pro-region sequences, operably linked to DNA sequences encoding proteins of interest and the like.

In certain aspects, the disclosure provides recombinant Gram-positive bacterial strains expressing one or more introduced polynucleotides encoding a protein of interest. In certain other aspects, the disclosure provides, compositions and methods for the design/construction of recombinant Gram-positive bacterial strains expressing one or more introduced novel polynucleotide constructs encoding proteins of interest, compositions and methods for cultivating recombinant strains expressing proteins of interest, compositions and methods for the enhanced production of proteins of interest and the like.

### I. Definitions

In view of the recombinant polynucleotides, recombinant (modified) strains and methods thereof described herein, the following terms and phrases are defined. Terms not defined herein should be accorded their ordinary meaning as used in the art.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described. All publications and patents cited herein are incorporated by reference in their entirety.

It is further noted that the claims may be drafted to exclude any optional element. As such, this statement is intended to serve as antecedent basis for use of such exclusive terminology as “solely,” “only”, “excluding”, “not including” and the like, in connection with the recitation of claim elements, or use of a “negative” limitation or proviso thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the phrases “Gram-positive bacteria”, Gram-positive cells” “Gram-positive bacterial strains”, and/or “Gram positive bacterial cells” have the same meaning as used in the art. For example, Gram-positive bacterial cells include all strains of Actinobacteria and Firmicutes. In certain embodiments, such Gram-positive bacteria are of the classes Bacilli, Clostridia and Mollicutes.

As used herein, the genus “Bacillus” includes all species within the genus “Bacillus” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

As used herein, the terms “recombinant” or “non-natural” refer to an organism, microorganism, cell, nucleic acid molecule, or vector that has at least one engineered genetic alteration, or has been modified by the introduction of a heterologous nucleic acid molecule, or refer to a cell (e.g., a microbial cell) that has been altered such that the expression of a heterologous or endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions or other functional alteration of a cell's genetic material. For example, recombinant cells may express genes or other nucleic acid molecules that are not found in identical or homologous form within a native (wild-type) cell (e.g., a fusion or chimeric protein), or may provide an altered expression pattern of endogenous genes, such as being over-expressed, under-expressed, minimally expressed, or not expressed at all. “Recombination”, “recombining” or generating a “recombined” nucleic acid is generally the assembly of two or more nucleic acid fragments wherein the assembly gives rise to a chimeric gene.

The term “derived” encompasses the terms “originated” “obtained,” “obtainable,” and “created,” and generally indicates that one specified material or composition finds its origin in another specified material or composition, or has features that can be described with reference to another specified material or composition. For example, recombinant Gram-positive bacterial cells of the disclosure may be derived/obtained from any known Gram-positive bacterial strains.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein.

It is understood that the polynucleotides (or nucleic acid molecules) described herein include “genes”, “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including 5-untranslated regions (UTRs), and 3′-UTRs, as well as the coding sequence.

As used herein, an “endogenous gene” refers to a gene in its natural location in the genome of an organism.

As used herein, a “heterologous” gene, a “non-endogenous” gene, or a “foreign” gene refer to a gene not normally found in the host organism, but that is introduced into the host organism by gene transfer. The term “foreign” gene(s) comprises native genes inserted into a non-native organism and/or chimeric genes inserted into a native or non-native organism.

As used herein, a “heterologous control sequence”, refers to a gene expression control sequence (e.g., promoters, enhancers, terminators, etc.) which does not function in nature to regulate (control) the expression of the gene of interest. Generally, heterologous nucleic acids are not endogenous (native) to the cell, or a part of the genome in which they are present, and have been added to the cell, by infection, transfection, transduction, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any steps involved in the production of the polypeptide including, but not limited to, transcription, post-transcriptional modification, translation, post-translational modification, secretion and the like.

As used herein, the term “coding sequence” (CDS) refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

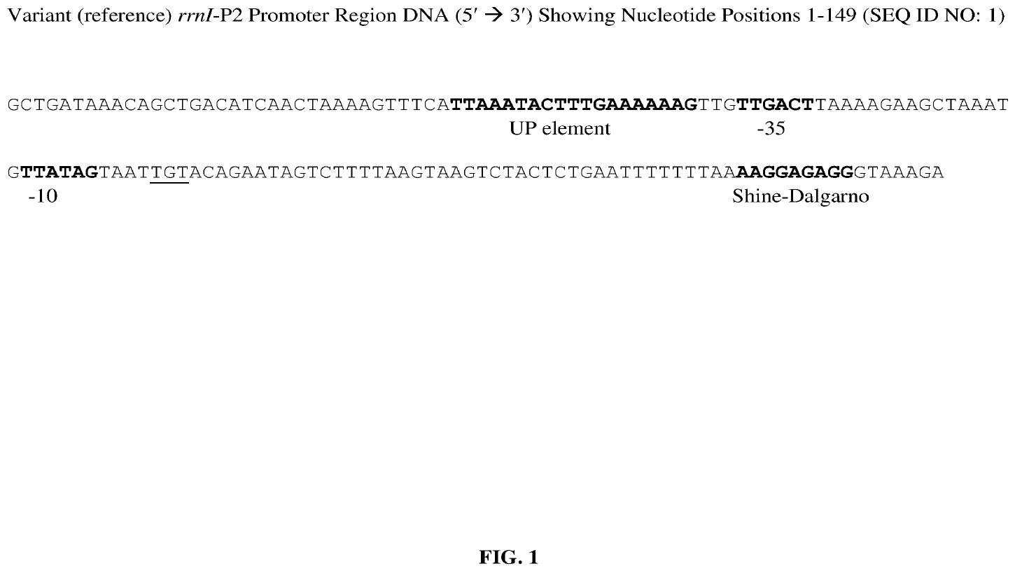

As used herein, the terms “promoter”, “promoter element”, “promoter sequence” and the like, refer to a nucleic acid (DNA) sequence capable of controlling the transcription of a gene coding sequence (CDS) into messenger RNA (mRNA) when the promoter region sequence is placed upstream (5′) and operably linked to the downstream (3′) gene CDS. As generally understood by of skilled in the art, promoters typically provide a site for specific binding by RNA polymerase and the initiation of transcription. In certain aspects, the term “promoter” refers to the minimal portion of the promoter nucleic acid sequence required to initiate transcription (i.e., comprising RNA polymerase binding sites). For example, a promoter generally comprises a “−10” (consensus sequence) element and a “−35” (consensus sequence) element, which are upstream (5′) and relative to the +1 transcription start site (TSS) of the gene CDS to be transcribed. The core promoter −10 and −35 elements are generally referred to in the art as the “TATAAT” (Pribnow box) consensus region and the “TTGACA” consensus region, respectively. The spacing of the core promoter (−10 and −35) regions are generally separated (spaced) by about fifteen-twenty (15-20) intervening base pairs (nucleotides) as shown in FIG. 1.

Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters can be constitutive promoters, inducible promoters, tunable promoters, hybrid promoters, synthetic promoters, tandem promoters, etc. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

In certain aspects, an upstream (5′) promoter sequence (pro) operably linked to a downstream DNA sequence encoding a protein signal sequence (SS) operably linked to a downstream DNA sequence encoding a pro-region sequence (PRO) operably linked to a downstream (3′) DNA sequence (ORF) encoding a mature protein of interest, may be schematically presented as, 5′-[pro]-[SS]-[PRO]-[ORF]-3′.

As used herein, a “functional promoter sequence controlling the expression of a gene of interest linked to the gene of interest's protein coding sequence” refers to a promoter sequence which controls the transcription and translation of the coding sequence in a desired Gram-positive host cell. For example, in certain embodiments, the present disclosure provides polynucleotides comprising an upstream (5′) promoter (or 5′ promoter region, or tandem 5′ promoters and the like) functional in a Gram-positive cell, wherein the functional promoter region is operably linked to a nucleic acid sequence encoding a protein of interest.

As used herein, the term “precursor protein” refers to an inactive form of a protein. In certain aspects, a full-length protein is synthesized as precursor, in the form of a pro-sequence and mature protein (abbreviated, “pre-protein”). In other aspects, a full-length protein is synthesized as precursor, in the form of a signal peptide sequence, a pro-sequence and mature protein (abbreviated, “pre-pro-protein”). For example, pre-sequences usually act as signal peptides for transport, and pro-sequences are typically essential for the correct folding of the associated (mature) protein.

As used herein, the term “mature protein” refers to an active form of a protein, in contrast to the inactive precursor (full-length) protein.

As used herein, the terms “signal sequence”, “secretion signal” and “signal peptide” may be used interchangeably and refer to a sequence of amino acid residues that may participate in the secretion or direct transport of a precursor protein. The signal (pre) sequence is typically cleaved from the precursor protein by a signal peptidase during translocation. The signal (pre) sequence is typically located N-terminal to the mature protein sequence, or located N-terminal to the pro-region (pro) sequence when a signal (pre) sequence and a pro-region (pro) sequence are used in operable combination and upstream (5′) of the mature POI sequence.

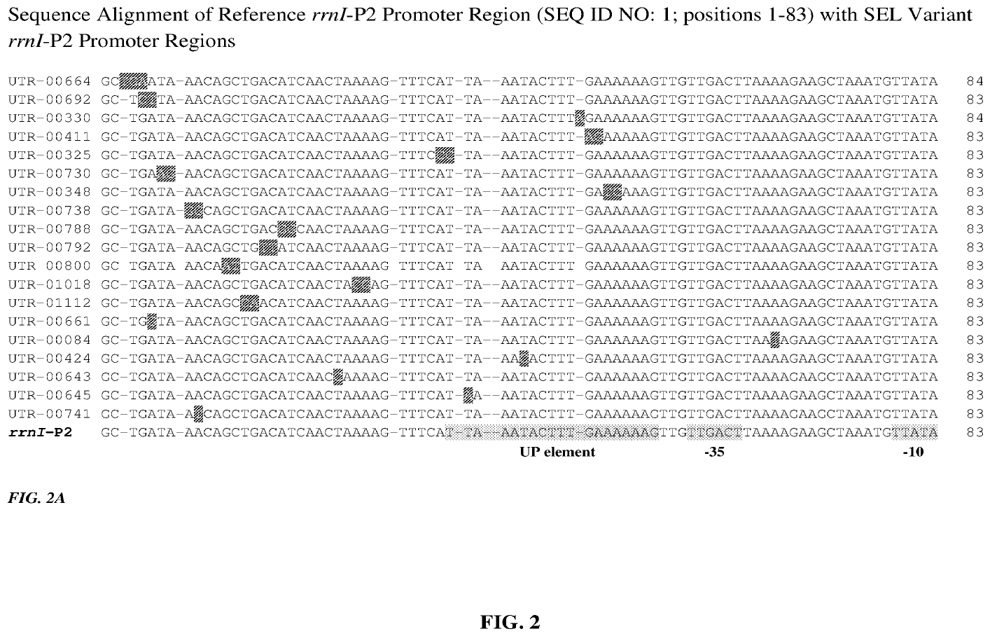

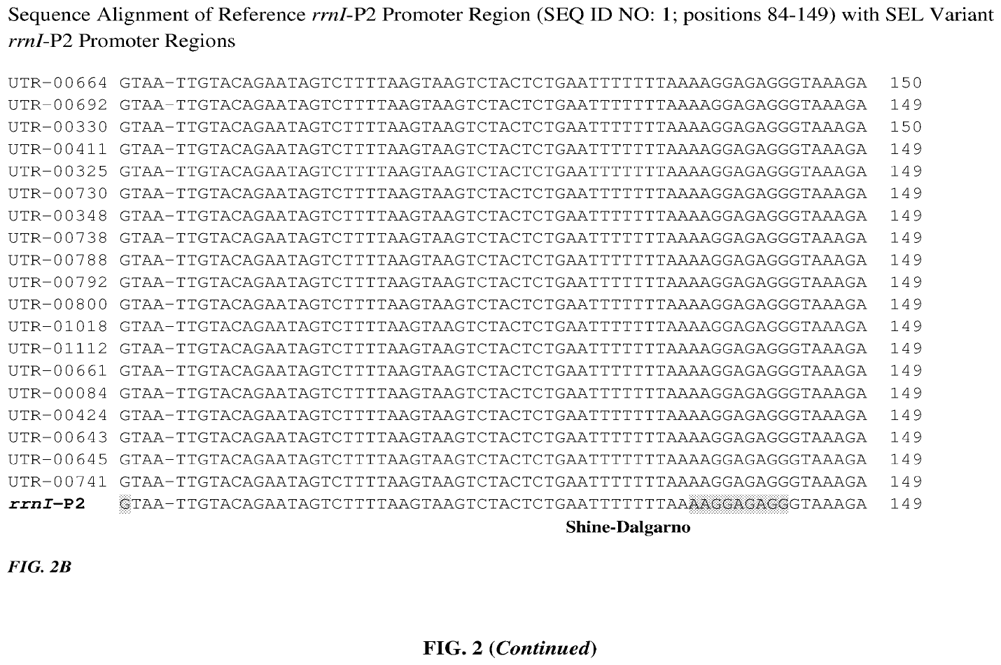

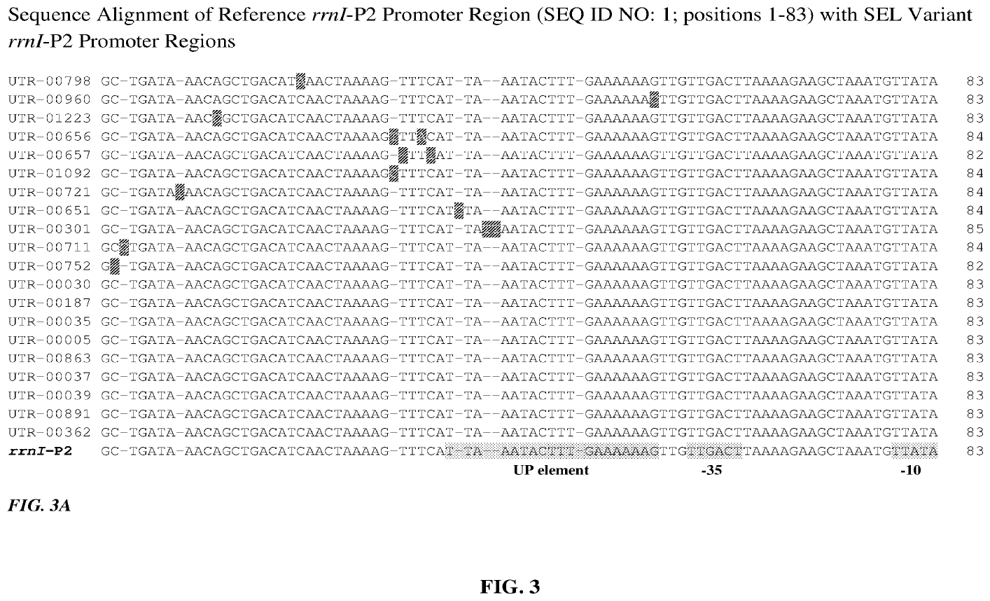

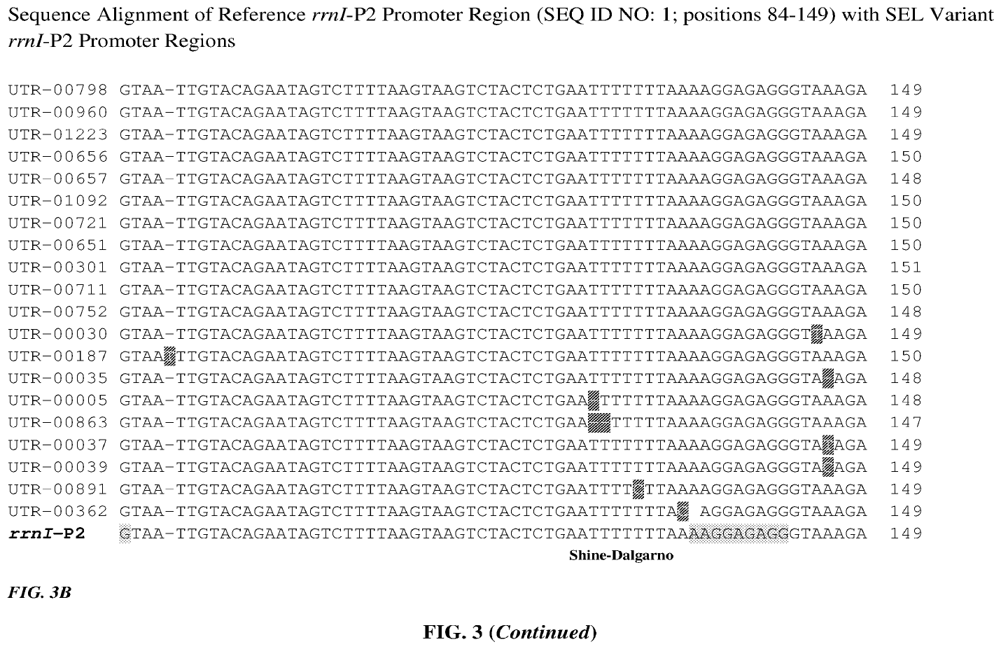

As used herein, phrases such as “variant rrnI-P2 promoter/5′-UTR region” and/or “reference rrnI-P2 promoter/5′-UTR region” particularly refer to the variant B. subtilis rrnI-P2 promoter/5′-UTR region DNA sequence set forth in SEQ ID NO: 1 (e.g., see FIG. 1). Thus, in certain aspects, the variant rrnI-P2 promoter/5′-UTR region sequence (SEQ ID NO: 1) may be referred to as a reference sequence, or a control sequence, particularly when being compared with one or more SEL variant promoter/5′-UTR region sequences of the disclosure. For example, as presented in FIG. 2 and FIG. 3, the reference rrnIp2 promoter/5′-UTR region sequence (nucleotide positions 1-149) has been aligned with certain site evaluation library (SEL) variant promoter/5′-UTR region sequences of the disclosure. More particularly, the SEL variant promoter/5′-UTR region sequences (e.g., see TABLE 2; SEQ ID NO: 8-48) are aligned with the reference rrnIp2 promoter/5′-UTR region sequence, wherein nucleotide positions 1-83 of the reference promoter/5′-UTR region sequence and the SEL variants are shown in FIG. 2A and FIG. 3A, and nucleotide positions 84-149 of the reference promoter/5′-UTR region sequence and the same SEL variants are shown in FIG. 2B and FIG. 3B, respectively. As annotated in FIG. 1B, DNA sequence elements of the reference promoter/5′-UTR region sequence (SEQ ID NO: 1) include (5′ to 3′ direction) an upstream (UP) element, a −35 element, a −10 element and a Shine-Dalgarno (SD) element, which are indicated with bold nucleotides.

In certain aspects, a promoter comprises nucleotides which are upstream (5′) of the promoter, wherein such upstream (5′) nucleotides are referred to herein as an “upstream promoter element” (abbreviated, “UP element” or “UP sequence”). Thus, as used herein, a “UP sequence” refers to an “A+T” rich (nucleic acid) sequence region located upstream of the −35 core promoter element. The UP sequence may be further described as a nucleic acid sequence region located upstream of the −35 core promoter element, which UP sequence interacts directly with the C-terminal domain of the α-subunit of RNA polymerase. Thus, in certain embodiments, a promoter comprises one (or more) UP sequences positioned upstream and operably linked to a promoter.

As used herein, the phrase “transcription initiation site” (abbreviated “TIS”) generally refers to the base pair where transcription initiates. By convention, the transcription initiation site (TIS) in a DNA sequence of a transcription unit is numbered with nucleotide positions extending in the direction of transcription (i.e., 3′; downstream) being assigned positive “(+)” numbers, and the nucleotide positions extending in the opposite direction (i.e., 5′; upstream) are assigned negative “(−)” numbers.

As used herein, the phrases “translation start site” (abbreviated, “tss”) and “translation start site (tss) codon” may be used interchangeably, and refer to a three (3) nucleotide translation start site (tss) codon. For example, a prokaryotic “tss codon” includes, but is not limited to, “AUG”, “GUG”, “UGG”, and the like.

As used herein, the term “Shine-Dalgarno” sequence (abbreviated, “SD” sequence) means a messenger RNA (mRNA) ribosomal binding site, generally located around 8 nucleotides upstream (5′) of the start codon (e.g., AUG). As understood in the art, the SD sequence helps recruit the ribosome to the mRNA to initiate protein synthesis by aligning the ribosome with the start codon (e.g., AUG), wherein transfer RNA (t-RNA) may add amino acids in sequence as dictated by the codons, moving downstream (3′) from the translational start site (tss).

As used herein, the terms “pro sequence”, “pro-sequence” and “pro-region sequence” may be used interchangeably and abbreviated as “PRO” sequence, “Pro” sequence, “pro” sequence and the like. The term pro-sequence as used herein has the same meaning as understood in the art. For example, the B. subtilis alkaline serine protease “subtilisin” is first produced as a pre-pro-subtilisin, which consists of a signal (pre) sequence for protein secretion followed by a seventy-seven (77) amino acid pro-region (pro) sequence followed by the amino acid sequence encoding the mature subtilisin (e.g., pre-pro-subtilisin). Pro-sequences are often essential for the correct folding of the associated (mature) protein, acting as an intra-molecular chaperone (e.g., catalyzing the protein-folding reaction directly). Likewise, pro-sequences may be required for both folding and intracellular transport (or secretion) of the mature protein of interest, suggesting that these two functionalities are intimately related. In certain aspects, a pro-region sequence of the disclosure comprises an amino acid sequence derived from a wild-type (WT, reference) B. lentus pro-region sequence of SEQ ID NO: 5.

As used herein, the phrase “polynucleotide encoding a full-length protein” refers to a DNA sequence encoding a “precursor” protein; and the phrase “polynucleotide encoding a mature protein” refers to a DNA sequence encoding a “mature” protein, as defined herein. In certain aspects, a polynucleotide encoding a precursor protein comprises at least an upstream (5′) DNA sequence encoding pro-region amino acid sequence operably linked to a downstream (3′) DNA sequence (e.g., open reading frame, ORF) encoding the amino acid sequence of a mature protein of interest (POI). In other aspects, a polynucleotide encoding a precursor protein comprises at least an upstream (5′) DNA sequence encoding a protein signal sequence operably linked to a downstream (3′) DNA sequence encoding pro-region amino acid sequence operably linked to a downstream (3′) ORF encoding the amino acid sequence of a mature POI.

As used herein, the term “untranslated region” may be abbreviated “UTR”.

As used herein, the phrases “five prime (5′) untranslated region”, “5′ untranslated region” and/or “5′ transcript leader” may be used interchangeably and abbreviated as “5′-UTR”. As generally understood in the art, the 5′-UTR is known to be the region of a messenger RNA (mRNA) that is directly upstream (5′) from the initiation codon.

A nucleic acid is “operably linked” when it is placed into a functional relationship with another nucleic acid sequence. For example, DNA encoding a secretory leader (i.e., a signal sequence) is operably linked to DNA encoding a polypeptide if it is expressed as a pre-protein that participates in the secretion of the polypeptide; a promoter or enhancer is operably linked to a coding sequence (CDS, ORF) if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. Thus, the term operably linked generally refers to the association (juxtaposition) of nucleic acid sequences on a single nucleic acid fragment so that the function of one is affected by the other. For example, a promoter (pro) is operably linked to a gene coding sequence (gene CDS) if it controls the transcription of the gene CDS.

As used herein, a DNA encoding a “wild-type B. subtilis aprE signal peptide sequence” may be abbreviated “aprE SS”, and comprises the nucleotide sequence of SEQ ID NO: 4.

As used herein, a DNA encoding a “wild-type B. lentus pro-peptide region DNA sequence” may be abbreviated “PRO sequence”, “PRO region”, or “PRO”, and comprises the nucleotide sequence of SEQ ID NO: 5.

As used herein, a “wild-type B. amyloliquefaciens BPN′ terminator (BPN′ term)” may be abbreviated “term”, and comprises the nucleotide sequence of SEQ ID NO: 6.

As used herein, a wild-type Bacillus gibsonii (BG46) subtilisin comprising the amino acid sequence of SEQ ID NO: 2 is abbreviated “WT BG46”.

As used herein, a variant Bacillus gibsonii (BG46) subtilisin comprising the amino acid sequence of SEQ ID NO: 3 is abbreviated “BG46 variant”, which BG46 variant was derived from the wild-type B. gibsonii (BG46) subtilisin reporter protein (SEQ ID NO: 2).

As used herein, an “upstream (5′) aprE flanking region (FR) sequence” comprises SEQ ID NO: 49.

As used herein, a “downstream (3′) aprE flanking region (FR) sequences” comprises SEQ ID NO: 50, and includes a “kanamycin (Kan) gene expression cassette” for selection (SEQ ID NO: 7).

As used herein, exemplary proteases may be referred to as “reporter proteins”. In certain one or more embodiments of the disclosure, exemplary reporter proteins are expressed/produced by one or more recombinant (modified) cells of the disclosure. In certain embodiments, reporter proteins include, but are not limited to, native and variant Bacillus sp. subtilisins.

As used herein, the term “subtilisin” refers to any member of the S8 serine protease family as described in MEROPS—The Peptidase Data base (Rawlings et al., 2006). The term subtilisin includes a wide variety of Bacillus subtilisins which have been identified and sequenced e.g., subtilisin 168, subtilisin BPN′, subtilisin Carlsberg, etc., and includes mutant (variant) proteases derived therefrom and the like.

In certain one or more embodiments, exemplary subtilisin reporters include, but are not limited to, the native B. clausii subtilisin and functional variants thereof, the native B. gibsonii subtilisin and functional variants thereof, the native B. lentus subtilisin and functional variants thereof, the native B. licheniformis subtilisin (AprL) and functional variants thereof, the native B. subtilis subtilisin (AprE) and functional variants thereof, the native B. amyloliquefaciens subtilisin (BPN′) and functional variants thereof, and the like. In certain aspects, exemplary B. clausii, B. gibsonii and/or B. lentus subtilisin reporters may be referred to as alkaline proteases. For instance, alkaline subtilisins generally have an isoelectric point (pI) of about 9.5, whereas the B. licheniformis, B. subtilis and B. amyloliquefaciens subtilisins have a pI of about 6.5.

In certain embodiments, the disclosure is related to one or more variant subtilisins derived from a parent (native) subtilisin sequence, such as the native B. subtilis subtilisin (e.g., 168), the native B. amyloliquefaciens (e.g., BPN′), the native B. lichenformis subtilisin (e.g., Carlsberg), the native B. lentus subtilisin (e.g., 309), the B. alcalophilus subtilisin (e.g., PB92) and the like. One of skill may readily design, construct, screen, and identity functional subtilisin variants using routine methods known in the art. In particular, PCT Publication Nos. WO2010/056634, WO2011/130222, WO2015/089447, WO2016/202839, WO2017/207762 and WO2023/114936 (each incorporated herein by reference in its entirety) describe suitable methods and compositions for constructing functional subtilisin variants derived from a native B. clausii subtilisin, functional subtilisin variants derived from a native B. amyloliquefaciens subtilisin, functional subtilisin variants derived from a native B. gibsonii subtilisin and the like.

As used herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, transcription leader sequences, RNA processing site, effector binding site and stem-loop structures.

As used herein, a “host cell” refers to a cell that has the capacity to act as a host or expression vehicle for a newly introduced DNA sequence. This, in certain embodiments of the disclosure, the host cells are Gram-positive cells (e.g., Bacillus sp.) and/or Gram-negative cells (e.g., E. coli).

As used herein, a “modified cell” refers to a recombinant cell that comprises at least one genetic modification which is not present in the parental, reference or control cell from which the modified cell is derived.

As used herein, when the expression and/or production of a protein of interest (POI) in a recombinant (modified) cell is being compared to the expression and/or production of the same POI in an unmodified (control) cell, it will be understood that the modified and unmodified cells are grown/cultivated/fermented under the same conditions (e.g., the same conditions such as media, temperature, pH and the like).

As used herein, an “increased amount”, when used in phrases such as a “recombinant cell ‘expresses/produces an increased amount’ of a protein of interest relative to the unmodified (control) cell”, particularly refers to an “increased amount” of a protein of interest (POI) expressed/produced in by the recombinant cell, which “increased amount” is always relative to the unmodified (control) cells expressing/producing the same POI, wherein the modified and unmodified cells are grown/cultured/fermented under the same conditions.

As used herein, “increasing” protein production or “increased” protein production is meant an increased amount of protein produced (e.g., a protein of interest). The protein may be produced inside the host cell, or secreted (or transported) into the culture medium. In certain embodiments, the protein of interest is produced (secreted) into the culture medium. Increased protein production may be detected for example, as higher maximal level of protein or enzymatic activity (e.g., such as amylase activity), or total extracellular protein produced as compared to the parental host cell.

As used herein, the terms “modification” and “genetic modification” are used interchangeably and include: (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein.

As used herein, the term “introducing”, as used in phrases such as “introducing into a Gram-positive bacterial cell a ‘gene’, a ‘polynucleotide’, an ‘open reading frame’ (ORF), a ‘gene coding sequence, a ‘vector’, an ‘expression cassette’”, and the like, includes methods known in the art for introducing polynucleotides (DNA) into a cell, including, but not limited to protoplast fusion, natural or artificial transformation (e.g., calcium chloride, electroporation), transduction, transfection, conjugation and the like.

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). Transformation therefore generally refers to introducing an exogenous DNA into a host cell so that the DNA is maintained as a chromosomal integrant or a self-replicating extra-chromosomal vector.

As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein, “disruption of a gene” or a “gene disruption”, are used interchangeably and refer broadly to any genetic modification that substantially prevents a host cell from producing a functional gene product (e.g., a protein). Thus, as used herein, a gene disruption includes, but is not limited to, frameshift mutations, premature stop codons (i.e., such that a functional protein is not made), substitutions eliminating or reducing activity of the protein internal deletions (such that a functional protein is not made), insertions disrupting the coding sequence, mutations removing the operable link between a native promoter required for transcription and the open reading frame, and the like.

As used herein “an incoming sequence” refers to a DNA sequence that is introduced into the bacterial cell chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein, “homology box” refers to a nucleic acid sequence, which is homologous to a sequence in the bacterial cell chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the bacterial cell chromosome a DNA construct is integrated and directs what part of the chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, a host cell “genome”, a bacterial (host) cell “genome”, or a Bacillus sp. (host) cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell. in some embodiments plasmids exist in a parental cell and are lost in the daughter cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously or can integrate into a chromosome of a host organism).

An “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. For example, in certain embodiments, a parental B. licheniformis (host) cell is modified (e.g., transformed) by introducing therein one or more “targeting vectors”.

As used herein, the term “protein of interest” or “POI” refers to a polypeptide of interest that is desired to be expressed in a modified (recombinant) Gram-positive host cell, wherein the POI is preferably expressed at increased levels (i.e., relative to the “unmodified” (parental or control) cell). Thus, as used herein, a POI may be an enzyme, a substrate-binding protein, a surface-active protein, a structural protein, a receptor protein, and the like. In certain embodiments, a modified cell of the disclosure produces an increased amount of a heterologous protein of interest relative to the control cell. In particular embodiments, an increased amount of a protein of interest produced by a modified cell of the disclosure is at least a 0.5% increase, at least a 1.0% increase, at least a 5.0% increase, or a greater than 5.0% increase, relative to the control cell.

Similarly, as defined herein, a “gene of interest” or “GOI” refers a nucleic acid sequence (e.g., a polynucleotide, a gene or an ORF) which encodes a POI. A “gene of interest” encoding a “protein of interest” may be a naturally occurring gene, a mutated gene or a synthetic gene.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, α-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof).

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide. Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence.

As used herein, a “variant” polynucleotide refers to a polynucleotide having a specified degree of sequence homology/identity with a parent polynucleotide, or hybridizes with a parent polynucleotide (or a complement thereof) under stringent hybridization conditions. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As used herein, the term “homology” relates to homologous polynucleotides or polypeptides. If two or more polynucleotides or two or more polypeptides are homologous, this means that the homologous polynucleotides or polypeptides have a “degree of identity” of at least 50%, at least 60%, more preferably at least 70%, even more preferably at least 85%, still more preferably at least 90%, more preferably at least 95%, and most preferably at least 98%.

The degree of homology between sequences can be determined using any suitable method known in the art (see, e.g., Smith and Waterman, 1981; Needleman and Wunsch, 1970; Pearson and Lipman, 1988; programs such as GAP, BESTFIT, FASTA, and TFASTA in the Wisconsin Genetics Software Package (Genetics Computer Group, Madison, WI); and Devereux et al., 1984). For purposes of the present invention, the degree of identity between two amino acid sequences is determined using the Needleman-Wunsch algorithm (Needleman and Wunsch, 1970) as implemented in the Needle program of the EMBOSS package (Rice et al., 2000), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

As used herein, the phrases “substantially similar” and “substantially identical”, in the context of at least two nucleic acids or polypeptides, typically means that a polynucleotide or polypeptide comprises a sequence that has at least about 40% identity, at least about 50% identity, at least about 60% identity, at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Sequence identity can be determined using known programs such as BLAST, ALIGN, and CLUSTAL using standard parameters.

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “specific productivity” is total amount of protein produced per cell per time over a given time period.

As used herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, the terms “modification” and “genetic modification” are used interchangeably and include: (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein.

### II. Novel Promoter Region Mutant Sequences

Certain Gram-positive bacterial promoters suitable for expressing proteins of interest have been described (e.g., Kim et al., 2008, U.S. Pat. No. 4,559,300, PCT Publication No. WO2013/086219 and PCT Publication No. WO2017/152169). As presented herein and set forth below in the Examples, Applicant has designed and constructed a site evaluation library (SEL) to test/screen genetic modifications (mutations) for enhanced productivity of recombinant proteins.

In particular, genetic modifications were performed on the promoter and 5′-UTR regions of expression constructs encoding an exemplary reporter protein, wherein the SEL constructs were introduced and expressed in recombinant Bacillus sp. cells. More particularly, as generally described in the Examples, a mature subtilisin reporter protein was expressed in B. subtills under the control of variant (mutant) promoter/5′-UTR region sequences (see, TABLE 2 and SEQ ID NO: 8-46) constructed by site scanning mutagenesis library (see, TABLE 1), or expressed under the control of the reference (control) rrnI-P2 promoter/5′-UTR region of SEQ ID NO: 1 (Example 1).

As described in Example 2, recombinant strains expressing the subtilisin reporter protein under the control of a mutant promoter/5′-UTR region sequence (TABLE 2) were compared to the reference strain expressing the same subtilisin reporter under the control of the (reference) rrnI-P2 promoter/5′-UTR region sequence (SEQ ID NO: 1). Likewise, as described in Example 3, performance index (PI) values of recombinant strains demonstrating increased protein productivity after seventy-two (72) hours of growth, as compared to the control strain (TABLE 3).

In particular, as described in Example 3, approximately 50% of the SEL variants constructed contain mutations upstream (5′) of the UP element, while about 22% of the SEL variants contain mutations in the UP element, as compared to the reference rrnI-P2 promoter/5′-UTR region of SEQ ID NO: 1 (see FIG. 2A and FIG. 3A). Approximately 22% of the SEL variants contain mutations around the Shine Dalgarno (SD) region, as compared to the reference rrnI-P2/5′-UTR region of SEQ ID NO: 1 (see, FIG. 2B and FIG. 3B). In contrast, about 72% of the SEL variants that contained mutations in the −35 and/or −10 promoter elements had performance index (PI) values between 0.1 and 0.5 after 24, 48 and 72 hours growth, and another 21% of the SEL variants that contained mutations in the SD region had PI values between 0.1 and 0.5 after 24, 48 and 72 hours growth (data not shown).

Thus, as generally set forth above, certain aspects of the disclosure are related to novel mutant promoter/5′-UTR region nucleic acids described herein. Certain embodiments are therefore related to such novel mutant promoter/5′-UTR region nucleic acid (DNA) sequences suitable for expressing gene coding sequences (CDS) of proteins of interest.

### III. Recombinant Polynucleotides and Molecular Biology

As generally set forth above, certain embodiments of the disclosure are related to novel mutant promoter/5′-UTR region sequences suitable for expressing a gene CDS encoding a proteins of interest. In related aspects, the disclosure provides recombinant polynucleotides comprising one or more mutant promoter/5′-UTR region nucleic acid (DNA) sequences. Thus, certain embodiments are related to recombinant polynucleotides (e.g., vectors, plasmids, expression cassettes, etc.), recombinant Gram-positive bacterial cells/strains expressing proteins of interest and the like. In certain aspects, the disclosure provides polynucleotide constructs suitable for introducing into recombinant Gram-positive bacterial cells (strains) for the enhanced production of proteins of interest. In certain aspects, a polynucleotide construct of the disclosure is referred to as an expression cassette, wherein the cassette comprises, in the 5′ to 3′ direction and in operable combination, at least an upstream (5′) a promoter/5′-UTR region DNA sequence linked to a downstream (3) gene CDS encoding a mature protein on interest (POI).

In certain aspects, expression cassettes comprise a variant promoter/5′-UTR region of the disclosure operably linked to a downstream gene CDS encoding a mature POI. In certain other aspects, expression cassettes of the disclosure comprise one or more DNA sequence elements, including, but not limited to, DNA sequence elements encoding protein/peptide signal (secretion) sequences (SS), DNA sequence elements (PRO) encoding pro-peptide (pro-region) amino acid residues, DNA sequence elements comprising transcriptional terminator sequences (term), DNA sequence elements comprising 5′-UTRs, 3′-UTRs, and the like.

As generally set forth above, certain embodiments of the disclosure are related to novel mutant pro-region sequences. In related aspects, the disclosure provides recombinant polynucleotides comprising one or more mutant pro-region nucleic acid (DNA) sequences. Thus, certain embodiments are related to recombinant polynucleotides (e.g., vectors, plasmids, expression cassettes, etc.), recombinant Gram-positive bacterial cells/strains expressing proteins of interest and the like. In certain aspects, the disclosure provides polynucleotide constructs suitable for introducing into recombinant Gram-positive bacterial cells (strains) for the enhanced production of proteins of interest. In certain aspects, a polynucleotide construct of the disclosure is referred to as an expression cassette, wherein the cassette comprises, in the 5′ to 3′ direction and in operable combination, at least an upstream (5′) a pro-region DNA sequence linked to a downstream (3′) gene CDS encoding a mature protein on interest (POI).

For example, one or more nucleic acid sequences described herein can be generated by using any suitable synthesis, manipulation, and/or isolation techniques, or combinations thereof. For example, one or more polynucleotides described herein may be produced using standard nucleic acid synthesis techniques, such as solid-phase synthesis techniques that are well-known to those skilled in the art. In such techniques, fragments of up to fifty (50) or more nucleotide bases are typically synthesized, then joined (e.g., by enzymatic or chemical ligation methods) to form essentially any desired continuous nucleic acid sequence. The synthesis of the one or more polynucleotide described herein can be also facilitated by any suitable method known in the art, including but not limited to chemical synthesis using the classical phosphoramidite method (e.g., Beaucage and Caruthers, 1981) or the method described by Matthes et al. (1984) as is typically practiced in automated synthetic methods. One or more polynucleotides described herein can also be produced by using an automatic DNA synthesizer. Customized nucleic acids can be ordered from a variety of commercial sources (e.g., ATUM (DNA 2.0), Newark, CA, USA; Life Tech (GeneArt), Carlsbad, CA, USA; GenScript, Ontario, Canada; Base Clear B. V., Leiden, Netherlands; Integrated DNA Technologies, Skokie, IL, USA; Ginkgo Bioworks (Gen9), Boston, MA, USA; and Twist Bioscience, San Francisco, CA, USA). Other techniques for synthesizing nucleic acids and related principles are described and known in the art.

Recombinant DNA techniques useful in modification of nucleic acids are well known in the art, such as, for example, restriction endonuclease digestion, ligation, reverse transcription and cDNA production, and polymerase chain reaction (e.g., PCR). One or more polynucleotides described herein may also be obtained by screening cDNA libraries using one or more oligonucleotide probes that can hybridize to or PCR-amplify polynucleotides which encode one or more variants described herein. Procedures for screening and isolating cDNA clones and PCR amplification procedures are well known to those of skill in the art and described in standard references known to those skilled in the art. One or more polynucleotides described herein can be obtained by altering a naturally occurring polynucleotide backbone (e.g., that encodes one or more variant pro-region sequences described herein) by, for example, a known mutagenesis procedure (e.g., site-directed mutagenesis, site saturation mutagenesis, and in vitro recombination). A variety of methods are known in the art that are suitable for generating modified polynucleotides described herein that encode one or more variants described herein, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches.

As generally set forth above and further described below in the Examples, certain embodiments of the disclosure are related to recombinant (modified) Gram-positive cells capable of producing increased amounts of heterologous proteins of interest. Certain embodiments are therefore related to methods for constructing such recombinant Gram-positive cells having increased protein production capabilities. In certain embodiments, one or more expression cassettes encoding a protein of intertest are introduced into Gram-positive cells of the disclosure. In exemplary embodiments, the cassettes are integrated into the genome of the cell. Thus, certain embodiments are related to nucleic acid molecules, polynucleotides (e.g., vectors, plasmids, expression cassettes), regulatory elements, and the like, suitable for use in constructing recombinant (modified) Gram-positive host cells.

Accordingly, as presented in the Examples and generally described herein, recombinant cells of the disclosure may be constructed by one of skill using standard and routine recombinant DNA and molecular cloning techniques well known in the art. Methods for genetic modification include, but are not limited to, (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion, (d) a gene deletion, (e) a gene down-regulation, (f) site specific mutagenesis and/or (g) random mutagenesis.

In certain embodiments, modified cells of the disclosure may be constructed by reducing or eliminating the expression of a gene, using methods well known in the art, for example, insertions, disruptions, replacements, or deletions. The portion of the gene to be modified or inactivated may be, for example, the coding region or a regulatory element required for expression of the coding region.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

In certain other embodiments a modified cell is constructed by gene deletion to eliminate or reduce the expression of the gene. Gene deletion techniques enable the partial or complete removal of the gene(s), thereby eliminating their expression, or expressing a non-functional (or reduced activity) protein product.

In such methods, the deletion of the gene(s) may be accomplished by homologous recombination using a plasmid that has been constructed to contiguously contain the 5′ and 3′ regions flanking the gene. The contiguous 5′ and 3′ regions may be introduced into a cell, for example, on a temperature-sensitive plasmid in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is affected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers. Thus, a person of skill in the art may readily identify nucleotide regions in the gene's coding sequence and/or the gene's non-coding sequence suitable for complete or partial deletion.

In other embodiments, a modified cell is constructed by introducing, substituting, or removing one or more nucleotides in the gene or a regulatory element required for the transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art. Thus, in certain embodiments, a gene of the disclosure is inactivated by complete or partial deletion.

In another embodiment, a modified cell is constructed by the process of gene conversion. For example, in the gene conversion method, a nucleic acid sequence corresponding to the gene(s) is mutagenized in vitro to produce a defective nucleic acid sequence, which is then transformed into the parental cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is affected by selection for the marker under conditions not permitting plasmid replication. Selection for a second recombination event leading to gene replacement is affected by examination of colonies for loss of the selectable marker and acquisition of the mutated gene. Alternatively, the defective nucleic acid sequence may contain an insertion, substitution, or deletion of one or more nucleotides of the gene, as described below.

In other embodiments, a modified cell is constructed by established anti-sense techniques using a nucleotide sequence complementary to the nucleic acid sequence of the gene. More specifically, expression of the gene by a Gram-positive cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to RNA interference (RNAi), small interfering RNA (siRNA), microRNA (miRNA), antisense oligonucleotides, and the like, all of which are well known to the skilled artisan.

In other embodiments, a modified cell is produced/constructed via CRISPR-Cas9 editing. For example, a gene encoding a protein of interest can be edited or disrupted (or deleted or down-regulated) by means of nucleic acid guided endonucleases, that find their target DNA by binding either a guide RNA (e.g., Cas9) and Cpf1 or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete the gene. For example, the gene encoding the nucleic acid guided endonuclease (for this purpose Cas9 from S. pyogenes) or a codon optimized gene encoding the Cas9 nuclease is operably linked to a promoter active in the Gram-positive cell and a terminator active in Gram-positive cells, thereby creating a Gram-positive cell Cas9 expression cassette. Likewise, one or more target sites unique to the gene of interest are readily identified by a person skilled in the art. For example, to build a DNA construct encoding a gRNA-directed to a target site within the gene of interest, the variable targeting domain (VT) will comprise nucleotides of the target site which are 5′ of the (PAM) proto-spacer adjacent motif (TGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a Gram-positive expression cassette for the gRNA is created by operably linking the DNA encoding the gRNA to a promoter active in Gram-positive cells and a terminator active in Gram-positive cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500 bp 5′ of targeted gene can be fused to about 500 bp 3′ of the targeted gene to generate an editing template, which template is used by the Gram-positive host's machinery to repair the DNA break generated by the RGEN.

The Cas9 expression cassette, the gRNA expression cassette and the editing template can be co-delivered to filamentous fungal cells using many different methods (e.g., protoplast fusion, electroporation, natural competence, or induced competence). The transformed cells are screened by PCR amplifying the target gene locus, by amplifying the locus with a forward and reverse primer. These primers can amplify the wild-type locus or the modified locus that has been edited by the RGEN. These fragments are then sequenced using a sequencing primer to identify edited colonies.

In yet other embodiments, a modified cell is constructed by random or specific mutagenesis using methods well known in the art, including, but not limited to, chemical mutagenesis and transposition. Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

PCT Publication No. WO2003/083125 discloses methods for modifying Gram-positive (Bacillus) cells, such as the creation of Bacillus deletion strains and DNA constructs using PCR fusion to bypass E. coli. PCT Publication No. WO2002/14490 discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion.

Those of skill in the art are well aware of suitable methods for introducing polynucleotide sequences into bacterial cells (e.g., Gram-negative cells, Gram-positive cells). Indeed, such methods as transformation including protoplast transformation and congression, transduction, and protoplast fusion are known and suited for use in the present disclosure. Methods of transformation are particularly preferred to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Bacillus strain by methods known in the art. In some embodiments, resolution of the vector from a host chromosome leaves the flanking regions in the chromosome, while removing the indigenous chromosomal region.

Promoters and promoter sequence regions for use in the expression of genes, coding sequences (CDS), open reading frames (ORFs) and/or variant sequences thereof in Gram-positive cells are generally known on one of skill in the art. Promoter sequences of the disclosure are generally chosen so that they are functional in the Gram-positive cells. For example, promoters useful for driving gene expression in Bacillus cells include, but are not limited to, the B. subtilis alkaline protease (aprE) promoter, the α-amylase promoter (amyE) of B. subtilis, the α-amylase promoter (amyL) of B. licheniformis, the α-amylase promoter of B. amyloliquefaciens, the neutral protease (nprE) promoter from B. subtilis, a mutant aprE promoter, or any other promoter from B licheniformis or other related Bacilli. Methods for screening and creating promoter libraries with a range of activities (promoter strength) in Bacillus cells is describe in Publication No. WO2002/14490.

### IV. Fermenting Gram-Positive Cells for the Production of Proteins

As generally described above, certain embodiments are related to compositions and methods for constructing and obtaining Gram-positive cells having increased protein production phenotypes. Thus, certain embodiments are related to methods of producing proteins of interest in Gram-positive cells by fermenting the cells in a suitable medium. Fermentation methods well known in the art can be applied to ferment Gram-positive cells of the disclosure.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Gram-positive cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Gram-positive cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

### V. Proteins of Interest

A protein of interest (POI) of the instant disclosure can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest.

For example, in certain embodiments, a modified Gram-positive cell of the disclosure produces at least about 0.1% more, at least about 0.5% more, at least about 1% more, at least about 5% more, at least about 6% more, at least about 7% more, at least about 8% more, at least about 9% more, or at least about 10% or more of a POI, relative to its unmodified (parental or control) cell.

In certain embodiments, a modified Gram-positive cell of the disclosure exhibits an increased specific productivity (Qp) of a POI relative the control cell. For example, the detection of specific productivity (Qp) is a suitable method for evaluating protein production. The specific productivity (Qp) can be determined using the following equation:

$$“ Qp = g P / g DCW \cdot hr ”$$

wherein, “gP” is grams of protein produced in the tank; “gDCW” is grams of dry cell weight (DCW) in the tank and “hr” is fermentation time in hours from the time of inoculation, which includes the time of production as well as growth time.

Thus, in certain other embodiments, a modified Gram-positive cell of the disclosure comprises a specific productivity (Qp) increase of at least about 0.1%, at least about 1%, at least about 5%, at least about 6%, at least about 7%, at least about 8%, at least about 9%, or at least about 10% or more, relative to the unmodified (parental) cell.

In certain embodiments, a POI or a variant POI thereof is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

Thus, in certain embodiments, a POI or a variant POI thereof is an enzyme selected from Enzyme Commission (EC) Number EC 1, EC 2, EC 3, EC 4, EC 5 or EC 6.

There are various assays known to those of ordinary skill in the art for detecting and measuring activity of intracellularly and extracellularly expressed proteins.

### VI. Exemplary Embodiments

Non-limiting embodiments of compositions and methods disclosed herein are as follows:
1. A variant nucleic acid (DNA) sequence comprising at least one mutation set forth in any one of SEQ ID NO: 8 through SEQ ID NO: 46, wherein the nucleotide positions of the variant nucleic acid sequence are numbered according to SEQ ID NO: 1.
2. A variant nucleic acid (DNA) sequence comprising any one of SEQ ID NO: 8 through SEQ ID NO: 46, wherein the nucleotide positions of the variant sequence are numbered according to SEQ ID NO: 1.
3. The variant nucleic acid of embodiment 1 or embodiment 2, comprising at least about 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 1.
4. The variant nucleic acid of any one of embodiments 1-3, further defined as a variant rrnI-P2 promoter and 5′-untranslated region (5′-UTR) sequence (variant rrnI-P2/5′-UTR sequence).
5. A polynucleotide comprising a variant nucleic acid sequence of any one of embodiments 1-4.
6. A polynucleotide comprising a variant nucleic acid sequence of any one of embodiments 1-4, operably linked to a downstream (3′) nucleic acid sequence encoding a protein of interest (POI).
7. A polynucleotide comprising a variant nucleic acid sequence of any one of embodiments 1-4, operably linked to a downstream (3′) nucleic acid sequence encoding a pro-region (PRO) operably linked to a downstream nucleic acid sequence encoding a protein of interest (POI).
8. A polynucleotide comprising a variant nucleic acid sequence of any one of embodiments 1-4, operably linked to a downstream (3′) nucleic acid sequence encoding a protein signal (secretion) sequence (SS) operably linked to a downstream nucleic acid sequence encoding a protein of interest (POI).
9. A polynucleotide comprising a variant nucleic acid sequence of any one of embodiments 1-4, operably linked to a downstream (3′) nucleic acid sequence a protein signal (secretion) sequence (SS) operably linked a downstream nucleic acid sequence encoding a pro-region (PRO) sequence operably linked to a downstream nucleic acid sequence encoding a protein of interest (POI).
10. The polynucleotide of any one of embodiments 5-9, further comprising a downstream (3′) terminator (term) sequence operably linked to the nucleic acid encoding the POI protein.
11. The polynucleotide of any one of embodiments 5-9, wherein the POI is selected from the group consisting of enzymes, antibodies, receptor proteins, lectins and regulatory proteins.
12. The polynucleotide of any one of embodiments 5-9, wherein the POI is an enzyme selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.
13. The polynucleotide of embodiment 12, wherein the enzyme is a protease.
14. The polynucleotide of embodiment 13, wherein the enzyme is a subtilisin.
15. The polynucleotide of embodiment 14, wherein the subtilisin comprises at least about 80% to 100% identity to SEQ ID NO: 2 or SEQ ID NO: 3.
16. The polynucleotide of embodiment 7 or embodiment 9, wherein the pro-region (PRO) sequence comprises at least about 80% to 100% identity to SEQ ID NO: 5.
17. The polynucleotide of embodiment 8 or embodiment 9, wherein the DNA encoding the protein signal (secretion) sequence (SS) comprises at least about 80% to 100% identity to SEQ ID NO: 4.
18. The polynucleotide of embodiment 10, wherein the DNA encoding the terminator (term) sequence comprises at least about 80% to 100% identity to SEQ ID NO: 6.
19. An expression cassette comprising a polynucleotide of any one of embodiments 5-18.
20. A Gram-positive bacterial cell comprising an introduced cassette of embodiment 19.
21. The Gram-positive cell of embodiment 20, wherein the cassette is integrated into the genome of the cell.
22. The Gram-positive cell of embodiment 20, wherein the cell is Bacillus sp. cell.
23. The Gram-positive cell of embodiment 22, wherein the cell is Bacillus sp. cell is selected from the group consisting of B. subtilis, B. lichenifornis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis.
25. A method for producing a protein of interest (POT) in a Gram-positive bacterial cell comprising (a) introducing into a Gram-positive bacterial cell a polynucleotide comprising an upstream (5′) variant promoter and 5′-untranslated region (5-UTR) nucleic acid sequence (i) comprising at least one mutation set forth in any one of SEQ ID NO: 8 through SEQ ID NO: 46, wherein the nucleotide positions of the variant promoter/5′-UTR sequence are numbered according to SEQ ID NO: 1, or (ii) comprising any one of SEQ ID NO: 8 through SEQ ID NO: 46, wherein the nucleotide positions of the variant promoter/5′-UTR sequence are numbered according to SEQ ID NO: 1, operably linked to a downstream (3′) open reading frame (ORF) encoding a protein of interest (POI), and (b) cultivating the modified cell under suitable conditions for the production of the POI.
26. A method for producing a protein of interest (POI) in a Gram-positive bacterial cell comprising (a) introducing into a Gram-positive bacterial cell a polynucleotide comprising an upstream (5′) variant promoter and 5′-untranslated region (5-UTR) nucleic acid sequence (i) comprising at least one mutation set forth in any one of SEQ ID NO: 8 through SEQ ID NO: 46, wherein the nucleotide positions of the variant promoter/5′-UTR sequence are numbered according to SEQ ID NO: 1, or (ii) comprising any one of SEQ ID NO: 8 through SEQ ID NO: 46, wherein the nucleotide positions of the variant promoter/5′-UTR sequence are numbered according to SEQ ID NO: 1, operably linked to a downstream (3′) nucleic acid sequence encoding a pro-region (PRO) operably linked to a downstream open reading frame (ORF) encoding a protein of interest (POI), and (b) cultivating the modified cell under suitable conditions for the production of the POI.
27. A method for producing a protein of interest (POI) in a Gram-positive bacterial cell comprising (a) introducing into a Gram-positive bacterial cell a polynucleotide comprising an upstream (5′) variant promoter and 5′-untranslated region (5-UTR) nucleic acid sequence (i) comprising at least one mutation set forth in any one of SEQ ID NO: 8 through SEQ ID NO: 46, wherein the nucleotide positions of the variant promoter/5′-UTR sequence are numbered according to SEQ ID NO: 1, or (ii) comprising any one of SEQ ID NO: 8 through SEQ ID NO: 46, wherein the nucleotide positions of the variant promoter/5′-UTR sequence are numbered according to SEQ ID NO: 1, operably linked to a downstream (3′) nucleic acid sequence encoding a protein signal (secretion) sequence (SS) operably linked to a downstream open reading frame (ORF) encoding a protein of interest (POI), and (b) cultivating the modified cell under suitable conditions for the production of the POI.
28. A method for producing a protein of interest (POI) in a Gram-positive bacterial cell comprising (a) introducing into a Gram-positive bacterial cell a polynucleotide comprising an upstream (5′) variant promoter and 5′-untranslated region (5-UTR) nucleic acid sequence (i) comprising at least one mutation set forth in any one of SEQ ID NO: 8 through SEQ ID NO: 46, wherein the nucleotide positions of the variant promoter/5′-UTR sequence are numbered according to SEQ ID NO: 1, or (ii) comprising any one of SEQ ID NO: 8 through SEQ ID NO: 46, wherein the nucleotide positions of the variant promoter/5′-UTR sequence are numbered according to SEQ ID NO: 1, operably linked to a downstream (3′) nucleic acid encoding a protein signal (secretion) sequence (SS) operably linked a downstream nucleic acid sequence encoding a pro-region (PRO) sequence operably linked to a downstream open reading frame (ORF) encoding a protein of interest (POI), and (b) cultivating the modified cell under suitable conditions for the production of the POI.
29. The method of any one of embodiments 25-28, wherein the variant promoter/5′-UTR sequence comprises at least about 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 1.
30. The method of any one of embodiments 25-29, further comprising a downstream (3′) terminator (term) sequence operably linked to the ORF encoding the POI.
31. The method of any one of embodiments 25-29, wherein the modified cell produces an increased amount of the POI relative to a control cell, cultivated under the same conditions.
32. The method of any one of embodiments 25-29, wherein the modified cell produces an increased amount of the POI relative to the control cell after about seventy-two (72) hours of cultivation.
33. The method of any one of embodiments 25-32, wherein the POI is selected from the group consisting of enzymes, antibodies, receptor proteins, lectins and regulatory proteins.
34. The method of any one of embodiments 25-32, wherein the POI is an enzyme selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.
35. The method of embodiment 34, wherein the enzyme is a protease.
36. The method of embodiment 35, wherein the protease is a subtilisin.
37. The method of embodiment 36, wherein the subtilisin comprises at least about 80% to 100% identity to SEQ ID NO: 2 or SEQ ID NO: 3.
38. The method of embodiment 26 or embodiment 28, wherein the pro-region (PRO) sequence comprises at least about 80% to 100% identity to SEQ ID NO: 5.
39 The method of embodiment 27 or embodiment 28, wherein the DNA encoding the protein signal (secretion) sequence (SS) comprises at least about 80% to 100% identity to SEQ ID NO: 4.
40. The method of embodiment 30, wherein the DNA encoding the terminator (term) sequence comprises at least about 80% to 100% identity to SEQ ID NO: 6.
41. The method of any one of embodiments 25-29, wherein the polynucleotide is integrated into the genome of the cell.
42. The method of any one of embodiments 25-29, wherein the Gram-positive cell is Bacillus sp. cell.
43. The method of embodiment 42, wherein Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis.

### EXAMPLES

Certain aspects of the present disclosure may be further understood in light of the following examples, which should not be construed as limiting. Modifications to materials and methods will be apparent to those skilled in the art. Standard recombinant DNA and molecular cloning techniques used herein are well known in the art (Ausubel et al., 1987; Sambrook et al., 1989).

### Example 1

### Insertion-Deletion Site Evaluation of Promoter/5′-Untranslated Region Sequences

In the instant example, a variant B. gibsonii (BG46 variant) subtilisin (SEQ ID NO: 3) was used as a reporter to monitor protein expression as described herein. More specifically, a DNA fragment comprising an upstream (5′) aprE gene flanking region (5′ aprE gene FR; SEQ ID NO: 49) was operably linked to a polynucleotide construct (expression cassette) comprising an upstream (5′) B. subtilis variant (control) rrnI-P2 promoter/5′-UTR region DNA sequence (SEQ ID NO: 1) operably linked to a DNA sequence (SEQ ID NO: 4) encoding an AprE signal sequence operably linked to a DNA sequence (SEQ ID NO: 5) encoding a B. lentus pro-peptide sequence operably linked to a DNA sequence encoding the mature BG46 variant reporter protein (SEQ ID NO: 3) operably linked to a B. amyloliquefaciens BPN′ terminator DNA sequence (SEQ ID NO: 6), which polynucleotide construct was operably linked to a downstream (3′) aprE gene flanking region (3′ aprE gene FR; SEQ ID NO: 50) sequence which includes a downstream kanamycin (kan) gene expression cassette. More particularly, this DNA fragment was assembled using standard molecular biology techniques and was used as template to develop linear DNA expression cassettes comprising one or more promoter region SEL mutations described herein.

### Insertion-Deletion Site Evaluation Libraries

As generally described herein, a seventy-five (75) insertion-deletion (In-Del) site evaluation library (SEL) was performed on the reference (control) rrnI-P2 promoter/5′-UTR region sequence (SEQ ID NO: 1), which SEL mutant promoter/5′-UTR region sequences were designed/constructed as generally described in TABLE 1, and developed as 4.4 kb fragments by Twist Bioscience HQ (South San Francisco). More particularly, linear DNA of the expression cassettes (TABLE 2) were used to transform competent B. subtilis cells, wherein the transformation mixtures were plated onto LA plates containing 1.8 ppm kanamycin and incubated overnight at 37° C. Single colonies were picked and grown in Luria broth at 37° C. under antibiotic selection.

DNA sequence analysis was performed to determine unique (variant) promoter/5′-UTR region sequences that were cherry picked into 96-well microtiter plates (MTPs). For example, as shown in FIG. 1, the reference rrnI-P2 promoter/5′-UTR region (SEQ ID NO: 1) comprises 149 nucleotides, wherein nucleotide positions are numbered 1-149 in the 5′ to 3′ direction. In particular, as shown in FIG. 1, the reference promoter/5′-UTR region comprises nucleotide positions 1-149 of SEQ ID NO: 1, which SEL resulted in seventy-five (75) unique promoter/5′-UTR region libraries by altering (modifying) two (2) adjacent nucleotide positions of SEQ ID NO: 1.

For example, TABLE 1 set forth below shows the thirty-one (31) possible variants of the reference promoter/5′-UTR region's first (1^{st}) site (i.e., adjacent nucleotide position 1 (guanine, “G”) and position 2 (cytosine, “C”) of SEQ ID NO: 1), wherein the 1^{st} column (TABLE 1) shows the two (2) nucleotide positions altered (“Variation”) relative to the two (2) nucleotides at the same positions of the reference rrnI-P2 p promoter/5′-UTR region (TABLE 1, 2^{nd} column; “Result w/ GC as reference”).

TABLE 1
Insertion-Deletion SEL Showing 31 Variants

| | | Result with GC as SEQ ID |
|---|---|---|
| Variation | Number (#) | NO: 1 (Reference) |
| AA | 1 | AA |
| AC | 2 | AC |
| AG | 3 | AG |
| AT | 4 | AT |
| CA | 5 | CA |
| CC | 6 | CC |
| CG | 7 | CG |
| CT | 8 | CT |
| GA | 9 | GA |
| GC | 10 | GC |
| GG | 11 | GG |
| GT | 12 | GT |
| TA | 13 | TA |
| TC | 14 | TC |
| TG | 15 | TG |
| TT | 16 | TT |
| Deletion of 1st nt | 17 | G |
| Deletion of 2nd nt | 18 | C |
| Double nt Deletion | 19 | — |
| A insertion after 1st nt | 20 | CAG |
| C insertion after 1st nt | 21 | CCG |
| G insertion after 1st nt | 22 | CGG |
| T insertion after 1st nt | 23 | CTG |
| A insertion after 2nd nt | 24 | CGA |
| C insertion after 2nd nt | 25 | CGC |
| G insertion after 2nd nt | 26 | CGG |
| T insertion after 2nd nt | 27 | CGT |
| AA insertion after 2nd nt | 28 | CGAA |
| GT insertion after 2nd nt | 29 | CGGT |
| TA insertion after 2nd nt | 30 | CGTA |
| TT insertion after 2nd nt | 31 | CGTT |

Likewise, TABLE 2 below presents the reference rrnI-P2 promoter/5′-UTR region and forty (40) mutant promoter/5′-UTR region sequences identified in the SEL (TABLE 2; UTR-00664, SEQ ID NO: 8 through UTR-00752, SEQ ID NO: 46). As further discussed in Example 2, in the reporter protein expression experiments, transformed cells were grown in 96-well MTPs in cultivation medium (enriched semi-defined media based on MOPs buffer, with urea as major nitrogen source, maltodextrin as the main carbon source, supplemented with 3% soytone for robust cell growth, containing antibiotic selection) for three (3) days at 32° C., 300 rpm, with 80% humidity in shaking incubator, which were centrifuged and filtrated. Clarified culture supernatants were used to measure (assay) reporter protease activity to determine productivity levels, wherein samples were taken after 72 hour timepoints (Example 3, TABLE 3). The reporter protease activity assay is further described below in Example 2.

TABLE 2
rrnI-P2 Promoter Region Variants

| Name | SEQ ID | DNA sequence |
|---|---|---|
| Control rrnI- | 1 | GCTGATAAACAGCTGACATCAACTAAAAGTTTCATTAAATAC |
| P2/5′-UTR | | TTTGAAAAAAGTTGTTGACTTAAAAGAAGCTAAATGTTATAG |
| | | TAATTGTACAGAATAGTCTTTTAAGTAAGTCTACTCTGAATT |
| | | TTTTTAAAAGGAGAGGGTAAAGA |
| UTR-00664 | 08 | GCTGAATAAACAGCTGACATCAACTAAAAGTTTCATTAAATA |
| | | CTTTGAAAAAAGTTGTTGACTTAAAAGAAGCTAAATGTTATA |
| | | GTAATTGTACAGAATAGTCTTTTAAGTAAGTCTACTCTGAATT |
| | | TTTTTAAAAGGAGAGGGTAAAGA |
| UTR-00692 | 09 | GCTCTTAAACAGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00330 | 10 | GCTGATAAACAGCTGACATCAACTAAAAGTTTCATTAAATAC |
| | | TTTAGAAAAAAGTTGTTGACTTAAAAGAAGCTAAATGTTATA |
| | | GTAATTGTACAGAATAGTCTTTTAAGTAAGTCTACTCTGAATT |
| | | TTTTTAAAAGGAGAGGGTAAAGA |
| UTR-00411 | 11 | GCTGATAAACAGCTGACATCAACTAAAAGTTTCATTAAATAC |
| | | TTTAGAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00325 | 12 | GCTGATAAACAGCTGACATCAACTAAAAGTTTCCCTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00730 | 13 | GCTGAAGAACAGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00348 | 14 | GCTGATAAACAGCTGACATCAACTAAAAGTTTCATTAAATAC |
| | | TTTGATCAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00738 | 15 | GCTGATAGGCAGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00788 | 16 | GCTGATAAACAGCTGACGGCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00792 | 17 | GCTGATAAACAGCTGTTATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00800 | 18 | GCTGATAAACAATTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-01018 | 19 | GCTGATAAACAGCTGACATCAACTACGAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-01112 | 20 | GCTGATAAACAGCCAACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00037 | 21 | GCTGATAAACAGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTATAGA |
| UTR-00039 | 22 | GCTGATAAACAGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTACAGA |
| UTR-00661 | 23 | GCTGGTAAACAGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00891 | 24 | CGCTGATAAACAGCTGACATCAACTAAAAGTTTCATTAAATA |
| | | CTTTGAAAAAAGTTGTTGACTTAAAAGAAGCTAAATGTTATA |
| | | GTAATTGTACAGAATAGTCTTTTAAGTAAGTCTACTCTGAATT |
| | | TTCTTAAAAGGAGAGGGTAAAGA |
| UTR-00084 | 25 | GCTGATAAACAGCTGACATCAACTAAAAGTTTCATTAAATAC |
| | | TTTGAAAAAAGTTGTTGACTTAATAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00362 | 26 | GCTGATAAACAGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTACAAGGAGAGGGTAAAGA |
| UTR-00424 | 27 | GCTGATAAACAGCTGACATCAACTAAAAGTTTCATTAAACAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00643 | 28 | GCTGATAAACAGCTGACATCAACCAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00645 | 29 | GCTGATAAACAGCTGACATCAACTAAAAGTTTCATAAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00741 | 30 | GCTGATAATCAGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00798 | 31 | GCTGATAAACAGCTGACATAAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00960 | 32 | GCTGATAAACAGCTGACATCAACTAAAAGTTTCATTAAATAC |
| | | TTTGAAAAAACTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-01223 | 33 | GCTGATAAACCGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00656 | 34 | GCTGATAAACAGCTGACATCAACTAAAAGTTTACATTAAATA |
| | | CTTTGAAAAAAGTTGTTGACTTAAAAGAAGCTAAATGTTATA |
| | | GTAATTGTACAGAATAGTCTTTTAAGTAAGTCTACTCTGAATT |
| | | TTTTTAAAAGGAGAGGGTAAAGA |
| UTR-00657 | 35 | CGCTGATAAACAGCTGACATCAACTAAAAGTTTATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAAGA |
| UTR-00030 | 36 | GCTGATAAACAGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTTAAGA |
| UTR-01092 | 37 | GCTGATAAACAGCTGACATCAACTAAAAGCTTTCATTAAATA |
| | | CTTTGAAAAAAGTTGTTGACTTAAAAGAAGCTAAATGTTATA |
| | | GTAATTGTACAGAATAGTCTTTTAAGTAAGTCTACTCTGAATT |
| | | TTTTTAAAAGGAGAGGGTAAAGA |
| UTR-00721 | 38 | GCTGATATAACAGCTGACATCAACTAAAAGTTTCATTAAATA |
| | | CTTTGAAAAAAGTTGTTGACTTAAAAGAAGCTAAATGTTATA |
| | | GTAATTGTACAGAATAGTCTTTTAAGTAAGTCTACTCTGAATT |
| | | TTTTTAAAAGGAGAGGGTAAAGA |
| UTR-00651 | 39 | GCTGATAAACAGCTGACATCAACTAAAAGTTTCATCTAAATA |
| | | CTTTGAAAAAAGTTGTTGACTTAAAAGAAGCTAAATGTTATA |
| | | GTAATTGTACAGAATAGTCTTTTAAGTAAGTCTACTCTGAATT |
| | | TTTTTAAAAGGAGAGGGTAAAGA |
| UTR-00301 | 40 | GCTGATAAACAGCTGACATCAACTAAAAGTTTCATTATTAAT |
| | | ACTTTGAAAAAAGTTGTTGACTTAAAAGAAGCTAAATGTTAT |
| | | AGTAATTGTACAGAATAGTCTTTTAAGTAAGTCTACTCTGAA |
| | | TTTTTTTAAAAGGAGAGGGTAAAGA |
| UTR-00187 | 41 | GCTGATAAACAGCTGACATCAACTAAAAGTTTCATTAAATAC |
| | | TTTGAAAAAAGTTGTTGACTTAAAAGAAGCTAAATGTTATAG |
| | | TAATTTGTACAGAATAGTCTTTTAAGTAAGTCTACTCTGAATT |
| | | TTTTTAAAAGGAGAGGGTAAAGA |
| UTR-00035 | 42 | GCTGATAAACAGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTTAAAAGGAGAGGGTAAGA |
| UTR-00005 | 43 | GCTGATAAACAGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTTAAAAGGAGAGGGTAAAGA |
| UTR-00863 | 44 | GCTGATAAACAGCTGACATCAACTAAAAGTTTCATTAAATAC |
| | | TTTGAAAAAAGTTGTTGACTTAAAAGAAGCTAAATGTTATAG |
| | | TAATTGTACAGAATAGTCTTTTAAGTAAGTCTACTCTGAATTT |
| | | TTAAAAGGAGAGGGTAAAGA |
| UTR-00711 | 45 | GCGTGATAAACAGCTGACATCAACTAAAAGTTTCATTAAATA |
| | | CTTTGAAAAAAGTTGTTGACTTAAAAGAAGCTAAATGTTATA |
| | | GTAATTGTACAGAATAGTCTTTTAAGTAAGTCTACTCTGAATT |
| | | TTTTTAAAAGGAGAGGGTAAAGA |
| UTR-00752 | 46 | GTGATAAACAGCTGACATCAACTAAAAGTTTCATTAAATACT |
| | | TTGAAAAAAGTTGTTGACTTAAAAGAAGCTAAATGTTATAGT |
| | | AATTGTACAGAATAGTCTTTTAAGTAAGTCTACTCTGAATTTT |
| | | TTTAAAAGGAGAGGGTAAAGA |

### Example 2

### Protease Activity Assay

The protease activity of the reporter protein (BG46 variant) was determined by measuring the hydrolysis of the synthetic suc-AAPF-pNA peptide substrate. For the AAPF assay, the reagent solutions used were 100 mMv Tris pH 8.6, 10 mM CalC1₂, 0.005% Tween®-80 (Tris/Ca buffer) and 160 mM suc-AAPF-pNA in DMSO (suc-AAPF-pNA stock solution; Sigma: S-7388). To prepare a working solution, one (1) mL suc-AAPF-pNA stock solution was added to 100 mL Tris/Ca buffer and mixed. An enzyme sample was added to a microtiter plate (MTP) containing one (1) mg/mL suc-AAPF-pNA working solution and assayed for activity at 405 nm over three-five (3-5) minutes using a SpectraMax plate reader in kinetic mode at room temperature. The protease activity was expressed as mOD/minute. In particular, the protease activity of each variant constructed was measured and compared to the reference construct (rrnI-P2 promoter/5′-UTR region; SEQ ID NO: 1) that was grown in the same plate. By dividing the value of reference sample by the value of variant sample, the performance index (PI) was measured and is presented (TABLE 3) as described in Example 3.

### Example 3

Promoter and 5′-Untranslated Region Modifications that Enhance Protein Productivity

As described above, the mature subtilisin reporter protein (BG46 variant) was expressed in B. subtilis under the control of variant (mutant) promoter/5′-UTR region sequences constructed by site scanning mutagenesis library (SEL, Example 1), wherein the constructs were assayed for reporter protein activity (Example 2). As shown in the DNA sequence alignments of FIG. 2 and FIG. 3, variant promoter/5′-UTR region sequences that showed increased productivity after 72 hours growth and had a performance index (PI) of greater than (>) 1.2 compared to the reference (control) rrnI-P2 promoter/5′-UTR region sequence (SEQ ID NO: 1) are set forth below in TABLE 3.

For example, approximately 50% of the SEL variants constructed contain mutations upstream of the UP element, while about 22% of the SEL variants contain mutations in the UP element (FIG. 2A/FIG. 3A) as compared to the reference rrnI-P2 promoter/5′-UTR region (SEQ ID NO: 1). Likewise, approximately 22% of the SEL variants contain mutations in the 5′-UTR around the Shine Dalgarno (SD) region (FIG. 2B/FIG. 3B) as compared to the reference rrnI-P2 promoter/5′-UTR region (SEQ ID NO: 1).

In contrast, about 72% of the SEL variants that contained mutations in the −35/−10 promoter region had a PI value between 0.1 and 0.5 after 72 hours growth, and another 21% of the SEL variants that contained mutations in the 5′-UTR around the SD region had a PI value between 0.1 and 0.5 after 72 hours growth (data not shown).

TABLE 3
Performance Index (PI) Reporter Protein Productivity

| Name | SEQ ID | PI @24 h | PI @48 h | PI @72 h |
|---|---|---|---|---|
| Control rrnI-P2/5′-UTR | 1 | 1.0 | 1.0 | 1.0 |
| UTR-00664 | 08 | 1.5 | 1.3 | 1.3 |
| UTR-00692 | 09 | 1.3 | 1.5 | 1.3 |
| UTR-00330 | 10 | 1.4 | 1.4 | 1.2 |
| UTR-00411 | 11 | 1.3 | 1.3 | 1.2 |
| UTR-00325 | 12 | 1.3 | 1.4 | 1.3 |
| UTR-00730 | 13 | 1.3 | 1.3 | 1.3 |
| UTR-00348 | 14 | 1.3 | 1.3 | 1.2 |
| UTR-00738 | 15 | 1.3 | 1.3 | 1.2 |
| UTR-00788 | 16 | 1.3 | 1.4 | 1.2 |
| UTR-00792 | 17 | 1.3 | 1.4 | 1.3 |
| UTR-00800 | 18 | 1.3 | 1.3 | 1.2 |
| UTR-01018 | 19 | 1.4 | 1.2 | 1.2 |
| UTR-01112 | 20 | 1.3 | 1.3 | 1.2 |
| UTR-00037 | 21 | 1.4 | 1.3 | 1.3 |
| UTR-00039 | 22 | 1.3 | 1.2 | 1.2 |
| UTR-00661 | 23 | 1.2 | 1.3 | 1.2 |
| UTR-00891 | 24 | 1.4 | 1.3 | 1.2 |
| UTR-00084 | 25 | 1.2 | 1.4 | 1.3 |
| UTR-00362 | 26 | 1.3 | 1.3 | 1.2 |
| UTR-00424 | 27 | 1.5 | 1.3 | 1.2 |
| UTR-00643 | 28 | 1.4 | 1.4 | 1.2 |
| UTR-00645 | 29 | 1.2 | 1.4 | 1.3 |
| UTR-00741 | 30 | 1.2 | 1.2 | 1.2 |
| UTR-00798 | 31 | 1.4 | 1.3 | 1.3 |
| UTR-00960 | 32 | 1.4 | 1.5 | 1.2 |
| UTR-01223 | 33 | 1.4 | 1.4 | 1.3 |
| UTR-00656 | 34 | 1.3 | 1.3 | 1.3 |
| UTR-00657 | 35 | 1.3 | 1.3 | 1.2 |
| UTR-00030 | 36 | 1.3 | 1.2 | 1.3 |
| UTR-01092 | 37 | 1.2 | 1.3 | 1.2 |
| UTR-00721 | 38 | 1.3 | 1.4 | 1.2 |
| UTR-00651 | 39 | 1.2 | 1.4 | 1.2 |
| UTR-00301 | 40 | 1.4 | 1.3 | 1.3 |
| UTR-00187 | 41 | 1.2 | 1.2 | 1.2 |
| UTR-00035 | 42 | 1.4 | 1.3 | 1.2 |
| UTR-00005 | 43 | 1.2 | 1.3 | 1.4 |
| UTR-00863 | 44 | 1.4 | 1.5 | 1.2 |
| UTR-00711 | 45 | 1.3 | 1.4 | 1.2 |
| UTR-00752 | 46 | 1.3 | 1.4 | 1.2 |

### REFERENCES

U.S. Pat. No. 4,559,300
PCT Publication No. WO2002/14490
PCT Publication No. WO2003/083125
PCT Publication No. WO2013/086219
PCT Publication No. WO2017/152169
Ausubel et al., “Current Protocols in Molecular Biology”, published by Greene Publishing Assoc. and Wiley-Interscience (1987).
Kim et al., “Comparison of PaprE, PamyE, and PP43 promoter strength for β-galactosidase and staphylokinase expression in Bacillus subtilis”, Biotechnology and Bioprocess Engineering, 13:313, 2008.
Sambrook et al., “Molecular Cloning: A Laboratory Manual” Cold Spring Harbor Laboratory: Cold Spring Harbor, N.Y. (1989), (2001) and (2012).

1. A variant nucleic acid comprising at least one mutation set forth in any one of SEQ ID NO: 8 through SEQ ID NO: 46, wherein the nucleotide positions of the variant nucleic acid sequence are numbered according to SEQ ID NO: 1.

2. The variant nucleic acid of claim 2, comprising at least about 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 1.

3. A polynucleotide comprising a variant nucleic acid sequence of claim 1.

4. The polynucleotide of claim 3, operably linked to a downstream nucleic acid encoding a protein of interest (POI).

5. The polynucleotide of claim 3, operably linked to a downstream nucleic acid encoding a pro-region operably linked to a downstream nucleic acid encoding a protein of interest (POI).

6. The polynucleotide of claim 3, operably linked to a downstream nucleic acid encoding a protein signal sequence operably linked to a downstream nucleic acid encoding a protein of interest (POI).

7. The polynucleotide of claim 3, operably linked to a downstream nucleic acid encoding a protein signal sequence operably linked a downstream nucleic acid encoding a pro-region sequence operably linked to a downstream nucleic acid encoding a protein of interest (POI).

8. The polynucleotide of claim 3, further comprising a downstream terminator sequence operably linked to the nucleic acid encoding the POI protein.

9. The polynucleotide of claim 3, wherein the POI is selected from the group consisting of enzymes, antibodies, receptor proteins, lectins and regulatory proteins.

10. The polynucleotide of claim 2, wherein the POI is an enzyme selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

11. The polynucleotide of claim 10, wherein the enzyme is a protease.

12. The polynucleotide of claim 11, wherein the protease is a subtilisin.

13. An expression cassette comprising the polynucleotide of claim 3.

14. A Gram-positive bacterial cell comprising an introduced cassette of claim 13.

15. The Gram-positive cell of claim 14, wherein the cassette is integrated into the genome of the cell.

16. A method for producing a protein of interest (POI) in a Gram-positive bacterial cell comprising:
(a) introducing into a Gram-positive bacterial cell a polynucleotide comprising an upstream variant promoter and 5′-untranslated region (5-UTR) nucleic acid sequence:
(i) comprising at least one mutation set forth in any one of SEQ ID NO: 8 through SEQ ID NO: 46, wherein the nucleotide positions of the variant promoter/5′-UTR sequence are numbered according to SEQ ID NO: 1, or
(ii) comprising any one of SEQ ID NO: 8 through SEQ ID NO: 46, wherein the nucleotide positions of the variant promoter/5′-UTR sequence are numbered according to SEQ ID NO: 1,
operably linked to a downstream open reading frame (ORF) encoding a protein of interest (POI), and
(b) cultivating the modified cell under suitable conditions for the production of the POI.

17. The method of claim 16, wherein the variant promoter/5′-UTR sequence comprises at least about 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 1.

18. The method of claim 16, wherein the modified cell produces an increased amount of the POI relative to a control cell cultivated under the same conditions, wherein the control cell comprises an introduced polynucleotide comprising an upstream promoter/5′-UTR sequence of SEQ ID NO: 1 operably linked to a downstream ORF encoding the same POI as the modified cell.

19. The method of claim 16, wherein the modified cell produces an increased amount of the POI relative to the control cell after at least seventy-two (72) hours of cultivation.

20. The method of claim 19, wherein the increased amount of the POI is at least 5% relative to the control cell after at least seventy-two (72) hours of cultivation.

21. The method of claim 16, wherein the POI is selected from the group consisting of enzymes, antibodies, receptor proteins, lectins and regulatory proteins.

22. The method of claim 16, wherein the Gram-positive cell is Bacillus sp. cell.


SEQUENCE LISTING (48 sequences)

SEQ ID NO: 1 - DNA, 149 bp, Bacillus subtilis
  source 1..149: mol_type = other DNA
gctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 2 - protein, 269 aa, Bacillus gibsonii
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 3 - protein, 269 aa, synthetic construct
QQTVPWGITRVQAPAVHNRGITGSGVRVAILDSGISAHEDLNIRGGASFVPGEPTTADLNGHGTHVAGTVAALDNSIGVIGVAPNAELYAVKVLGANGRGSVSGIAQGLEWAATNNMHIANMSLGAEGPSSTLERAVNYATSRDVLVIAATGNNGSGSVGYPARYANAMAVGATDQNNRRANFSQYGTGIDIVAPGVNVQSTYPGNRYVSLNGTSMATPHVAGAAALVKQRYPSWNATQIRNHLKNTATNLGNSSEFGSGLVNAEAATR

SEQ ID NO: 4 - DNA, 87 bp, Bacillus subtilis
  source 1..87: mol_type = other DNA
gtgagaagcaaaaaattgtggatcagcttgttgtttgcgttaacgttaatctttacgatggcgttcagcaacatgtctgcgcaggct

SEQ ID NO: 5 - protein, 84 aa, Bacillus lentus
AEEAKEKYLIGFNEQEAVSEFVEQVEANDEVAILSEEEEVEIELLHEFETIPVLSVELSPEDVDALELDPAISYIEEDAEVTTM

SEQ ID NO: 6 - DNA, 247 bp, Bacillus amyloliquefaciens
  source 1..247: mol_type = other DNA
acataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 7 - DNA, 1761 bp, synthetic construct
  source 1..1761: mol_type = other DNA
tcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttgaaagttggcgtataacatagtatcgacggagccgataacgcctcactcctcacatcaacccgttacttctattgtaatcataaattcaaattcttagaaccaagctgtgttccgcacttttccacccttttaagcatggaaaccccgatcgctgggaaaactaacaatgtttggagtgatgcaaatgaaaaaaatagtggcagccatcgtggtaatcggtcttgtgtttatcgcatttttttatctttacagccgatcaggcgatgtgtatcaatcggtagacgcggatttgatcacactgtcttcaagcggccaggaagatatcgagattgaaaaaagacagcacgtcaaagatatgctggatattatgaatcagggaaaacaggtgaagacagaaaaaacatcagcccctgattacgaagggacaatcaagtttcataaagaccggtatgactcattcagactatggattgacggcagccagcaagccgtttttttgaaggatggcacatactacaaattaagcaaaaatgatacaaaggcgctgctaaatattattaaaaaagaagcaaaggattgaaaatgaaaaagcgaagctaaccgcttcgctttttcattttattggggcaaaatatctctcagtgcccgtctgagcattttccccgtcgcatttttcggaatatcgtcaagaaacgtaatggcggcaggccgcttgtattttgccagatgcttttcgcagtgctgcatgatgtcctcctctgttaccccagagcgtttcggcaccacatatccctttaccgcttccccgctttgggggtccggcacgccgatgacaaccgcctccttgacgtccggatggctgtacagcacctcctccacctcccgcggatacacattgtatcctcctacaatgatcatgtcttttttccggtcaacaatgtaaaaatagccgtcctcatcccgtcttgccaagtcccccgtataaagccacccgtctttta

SEQ ID NO: 8 - DNA, 150 bp, synthetic construct
  source 1..150: mol_type = other DNA
gctgaataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 9 - DNA, 149 bp, synthetic construct
  source 1..149: mol_type = other DNA
gctcttaaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 10 - DNA, 150 bp, synthetic construct
  source 1..150: mol_type = other DNA
gctgataaacagctgacatcaactaaaagtttcattaaatactttagaaaaaagttgttgacttaaaagaagctaaatgttatagtaattgtacagaatagtcttttaagtaagtctactctgaatttttttaaaaggagagggtaaaga

SEQ ID NO: 11 - DNA, 149 bp, synthetic construct
  source 1..149: mol_type = other DNA
gctgataaacagctgacatcaactaaaagtttcattaaatactttagaaaaagttgttgacttaaaagaagctaaatgttatagtaattgtacagaatagtcttttaagtaagtctactctgaatttttttaaaaggagagggtaaaga

SEQ ID NO: 12 - DNA, 149 bp, synthetic construct
  source 1..149: mol_type = other DNA
gctgataaacagctgacatcaactaaaagtttccctaaatactttgaaaaaagttgttgacttaaaagaagctaaatgttatagtaattgtacagaatagtcttttaagtaagtctactctgaatttttttaaaaggagagggtaaaga

SEQ ID NO: 13 - DNA, 149 bp, synthetic construct
  source 1..149: mol_type = other DNA
gctgaagaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 14 - DNA, 149 bp, synthetic construct
  source 1..149: mol_type = other DNA
gctgataaacagctgacatcaactaaaagtttcattaaatactttgatcaaagttgttgacttaaaagaagctaaatgttatagtaattgtacagaatagtcttttaagtaagtctactctgaatttttttaaaaggagagggtaaaga

SEQ ID NO: 15 - DNA, 149 bp, synthetic construct
  source 1..149: mol_type = other DNA
gctgataggcagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 16 - DNA, 149 bp, synthetic construct
  source 1..149: mol_type = other DNA
gctgataaacagctgacggcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 17 - DNA, 149 bp, synthetic construct
  source 1..149: mol_type = other DNA
gctgataaacagctgttatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 18 - DNA, 149 bp, synthetic construct
  source 1..149: mol_type = other DNA
gctgataaacaattgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 19 - DNA, 149 bp, synthetic construct
  source 1..149: mol_type = other DNA
gctgataaacagctgacatcaactacgagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 20 - DNA, 149 bp, synthetic construct
  source 1..149: mol_type = other DNA
gctgataaacagccaacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 21 - DNA, 149 bp, synthetic construct
  source 1..149: mol_type = other DNA
gctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtataga

SEQ ID NO: 22 - DNA, 149 bp, synthetic construct
  source 1..149: mol_type = other DNA
gctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtacaga

SEQ ID NO: 23 - DNA, 149 bp, synthetic construct
  source 1..149: mol_type = other DNA
gctggtaaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 24 - DNA, 150 bp, synthetic construct
  source 1..150: mol_type = other DNA
cgctgataaacagctgacatcaactaaaagtttcattaaatactttgaaaaaagttgttgacttaaaagaagctaaatgttatagtaattgtacagaatagtcttttaagtaagtctactctgaattttcttaaaaggagagggtaaaga

SEQ ID NO: 25 - DNA, 149 bp, synthetic construct
  source 1..149: mol_type = other DNA
gctgataaacagctgacatcaactaaaagtttcattaaatactttgaaaaaagttgttgacttaatagaagctaaatgttatagtaattgtacagaatagtcttttaagtaagtctactctgaatttttttaaaaggagagggtaaaga

SEQ ID NO: 26 - DNA, 149 bp, synthetic construct
  source 1..149: mol_type = other DNA
gctgataaacagctgacatcaactaaaagtttcattaaatactttgaaaaaagttgttgacttaaaagaagctaaatgttatagtaattgtacagaatagtcttttaagtaagtctactctgaatttttttacaaggagagggtaaaga

SEQ ID NO: 27 - DNA, 149 bp, synthetic construct
  source 1..149: mol_type = other DNA
gctgataaacagctgacatcaactaaaagtttcattaaacactttgaaaaaagttgttgacttaaaagaagctaaatgttatagtaattgtacagaatagtcttttaagtaagtctactctgaatttttttaaaaggagagggtaaaga

SEQ ID NO: 28 - DNA, 149 bp, synthetic construct
  source 1..149: mol_type = other DNA
gctgataaacagctgacatcaaccaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 29 - DNA, 149 bp, synthetic construct
  source 1..149: mol_type = other DNA
gctgataaacagctgacatcaactaaaagtttcataaaatactttgaaaaaagttgttgacttaaaagaagctaaatgttatagtaattgtacagaatagtcttttaagtaagtctactctgaatttttttaaaaggagagggtaaaga

SEQ ID NO: 30 - DNA, 149 bp, synthetic construct
  source 1..149: mol_type = other DNA
gctgataatcagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 31 - DNA, 149 bp, synthetic construct
  source 1..149: mol_type = other DNA
gctgataaacagctgacataaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 32 - DNA, 149 bp, synthetic construct
  source 1..149: mol_type = other DNA
gctgataaacagctgacatcaactaaaagtttcattaaatactttgaaaaaacttgttgacttaaaagaagctaaatgttatagtaattgtacagaatagtcttttaagtaagtctactctgaatttttttaaaaggagagggtaaaga

SEQ ID NO: 33 - DNA, 149 bp, synthetic construct
  source 1..149: mol_type = other DNA
gctgataaaccgctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 34 - DNA, 150 bp, synthetic construct
  source 1..150: mol_type = other DNA
gctgataaacagctgacatcaactaaaagtttacattaaatactttgaaaaaagttgttgacttaaaagaagctaaatgttatagtaattgtacagaatagtcttttaagtaagtctactctgaatttttttaaaaggagagggtaaaga

SEQ ID NO: 35 - DNA, 149 bp, synthetic construct
  source 1..149: mol_type = other DNA
cgctgataaacagctgacatcaactaaaagtttattaaatactttgaaaaaagttgttgacttaaaagaagctaaatgttatagtaattgtacagaatagtcttttaagtaagtctactctgaatttttttaaaaggagagggtaaaga

SEQ ID NO: 36 - DNA, 149 bp, synthetic construct
  source 1..149: mol_type = other DNA
gctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggttaaga

SEQ ID NO: 37 - DNA, 150 bp, synthetic construct
  source 1..150: mol_type = other DNA
gctgataaacagctgacatcaactaaaagctttcattaaatactttgaaaaaagttgttgacttaaaagaagctaaatgttatagtaattgtacagaatagtcttttaagtaagtctactctgaatttttttaaaaggagagggtaaaga

SEQ ID NO: 38 - DNA, 150 bp, synthetic construct
  source 1..150: mol_type = other DNA
gctgatataacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 39 - DNA, 150 bp, synthetic construct
  source 1..150: mol_type = other DNA
gctgataaacagctgacatcaactaaaagtttcatctaaatactttgaaaaaagttgttgacttaaaagaagctaaatgttatagtaattgtacagaatagtcttttaagtaagtctactctgaatttttttaaaaggagagggtaaaga

SEQ ID NO: 40 - DNA, 151 bp, synthetic construct
  source 1..151: mol_type = other DNA
gctgataaacagctgacatcaactaaaagtttcattattaatactttgaaaaaagttgttgacttaaaagaagctaaatgttatagtaattgtacagaatagtcttttaagtaagtctactctgaatttttttaaaaggagagggtaaaga

SEQ ID NO: 41 - DNA, 150 bp, synthetic construct
  source 1..150: mol_type = other DNA
gctgataaacagctgacatcaactaaaagtttcattaaatactttgaaaaaagttgttgacttaaaagaagctaaatgttatagtaatttgtacagaatagtcttttaagtaagtctactctgaatttttttaaaaggagagggtaaaga

SEQ ID NO: 42 - DNA, 148 bp, synthetic construct
  source 1..148: mol_type = other DNA
gctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaga

SEQ ID NO: 43 - DNA, 148 bp, synthetic construct
  source 1..148: mol_type = other DNA
gctgataaacagctgacatcaactaaaagtttcattaaatactttgaaaaaagttgttgacttaaaagaagctaaatgttatagtaattgtacagaatagtcttttaagtaagtctactctgaattttttaaaaggagagggtaaaga

SEQ ID NO: 44 - DNA, 147 bp, synthetic construct
  source 1..147: mol_type = other DNA
gctgataaacagctgacatcaactaaaagtttcattaaatactttgaaaaaagttgttgacttaaaagaagctaaatgttatagtaattgtacagaatagtcttttaagtaagtctactctgaatttttaaaaggagagggtaaaga

SEQ ID NO: 45 - DNA, 150 bp, synthetic construct
  source 1..150: mol_type = other DNA
gcgtgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 46 - DNA, 148 bp, synthetic construct
  source 1..148: mol_type = other DNA
gtgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 47 - DNA, 980 bp, synthetic construct
  source 1..980: mol_type = other DNA
ctgacgatattgcctcctgcttttccggccagaccatctttgtaattaatgccggaataagcaactttaatcaggacaccatccttcggcaaatcctctgttgatatggttttcacatggactgaaacatcatcggcatttttttctgcctgcaaggcttgaaataacgttgacattcggcacactccttttcatttatatcgtaaccgaagaacgttcaaaaaaccaaatcatcaagccgccattttcacttcgccggcacattgagacaataatggacaaatccggtatcctcttcatagccgttttgctcatacaagcttcttgccttccggttgtggtgctcagtctgaagtgttaaacattttgccccgttttgccctgcataatcctttgcggcagaaagcagccggccgccggctccctttgtacgcgcatgaggaacgacaaataagtcatttaatatgtatatccttttcattgacacagaagaaaacgttggatagagctgggtaaagcctatgaattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcgctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 48 - DNA, 1761 bp, synthetic construct; identical to SEQ ID NO: 7
  source 1..1761: mol_type = other DNA